(19) United States
(12) Patent Application Publication
(10) Pub. No.: US 2009/0202574 A1
(43) Pub. Date: Aug. 13, 2009

(54) METHODS OF IMMUNE OR HAEMATOLOGICAL ENHANCEMENT, INHIBITING TUMOUR FORMATION OR GROWTH, AND TREATING OR PREVENTING CANCER

(76) Inventors: Jagat Rakesh Kanwar, Palmerston North (NZ); Neill Ward Haggarty, Palmerston North (NZ); Kay Patricia Palmano, Palmerston North (NZ); Geoffrey Wayne Krissansen, Palmerston North (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 11/719,736

(22) PCT Filed: Nov. 18, 2005
(86) PCT No.: PCT/NZ05/00305

§ 371 Date: Apr. 24, 2009

Related U.S. Application Data
(60) Provisional application No. 60/635,814, filed on Nov. 19, 2004.

(51) Int. Cl. A61K 39/00; A61K 38/40; A61P 35/00; A61P 37/02

(52) U.S. Cl. 424/184.1; 514/12; 514/6

(57) ABSTRACT

The present invention relates to administration of metal ion-saturated lactoferrin, preferably bovine lactoferrin, preferably iron-saturated bovine lactoferrin, or a metal ion-saturated functional variant or fragment thereof to inhibit tumour formation or growth, maintain or improve one or both of the white blood cell count and red blood cell count, stimulate the immune system and treat or prevent cancer. The methods and medicinal uses of the invention may be carried out by employing dietary (as foods or food supplements), nutraceutical or pharmaceutical compositions. Compositions useful in the methods of the invention are also provided.

### FIELD OF THE INVENTION

The present invention relates to methods of immune or haematological enhancement, inhibiting tumour formation or growth, and treating or preventing cancer. In particular the present invention relates to administration of metal ion-saturated lactoferrin, preferably bovine lactoferrin, preferably iron-saturated bovine lactoferrin, or a metal ion-saturated functional variant or fragment thereof to inhibit tumour growth, maintain or improve one or both of the white blood cell count and red blood cell count, stimulate the immune system and treat or prevent cancer. The methods and medicinal uses of the invention may be carried out by employing dietary (as foods or food supplements), nutraceutical or pharmaceutical compositions. Compositions useful in the methods of the invention are also provided.

### BACKGROUND OF THE INVENTION

Bovine lactoferrin (bLf) is a single-chain iron-binding glycoprotein of 78 kDa which is present in bovine milk. It is a natural defence protein present in most secretions commonly exposed to normal flora including milk, colostrum, tears, nasal secretions, saliva, bile, pancreatic juice, intestinal mucus, and genital secretions. It is secreted by neutrophils and present at high levels at sites of bacterial infection. It is a multifunctional protein that may regulate iron absorption in the intestine, promote intestinal cell growth, protect against microbial infection, regulate myelopoiesis, regulate systemic immune responses, and can prevent the development of cancer (reviewed in Ward, et al., 2002; Brock, J H, 2002; Weinburg, E D, 2001; Conneely, O M, 2001; Tomita, et al., 2002 and Tsuda, et al., 2002).

It has previously been reported that tumours do not respond well to chemotherapy in all cases. For example, chemotherapy efficacy varies for cancer sufferers depending on the cancer type, the nature and doses of the drugs used for treatment, the mechanisms by which the drugs work, and the therapeutic regimes.

It is known in the field that cancers differ in their sensitivity to chemotherapy, from the usually and often sensitive (e.g lymphomas, acute lymphoblastic leukemia (ALL), chronic lymphocytic leukemia (CLL), Hodgkin's disease, intermediate and high grade non-Hodgkin's lymphoma, for example, diffuse large cell lymphoma, Burkitt's lymphoma, lymphoblastic lymphoma, choriocarcinoma, embryonal tumours, myelomatosis, oat cell carcinoma of bronchus, testicular carcinoma, Ewing's sarcoma, Wilms' tumor, skin cancer) where complete clinical cures can be achieved to the largely resistant (bladder cancer, esophageal cancer, non-small cell lung cancer, hepatocellular carcinoma, renal carcinoma, pancreatic carcinoma, head and neck cancer, cervical carcinoma, liver carcinoma, lung carcinomas that are not oat cell). It has previously been reported that EL-4 tumours larger than 0.3 cm in diameter become completely non-responsive to immunotherapy and anti-angiogenic therapy (Kanwar, et al., 1999 and Sun, et al., 2001).

Published International PCT Application WO 03/099323 reported that bovine lactoferrin was inferior to recombinant human lactoferrin in that it caused a lesser increase in the intestinal IL-18 levels and did not increase the serum levels of IL-18. It reported that bovine lactoferrin does not have the same biological activity or effect as human lactoferrin.

It would therefore be desirable to provide an improved or alternative method of inhibiting tumour growth using lactoferrin or to at least provide the public with a useful choice.

### SUMMARY OF THE INVENTION

Accordingly, one aspect of the present invention relates to a method of inhibiting tumour formation in a subject by inducing apoptosis in the subject, inducing apoptosis of tumour cells in the subject, inhibiting angiogenesis in the subject, inhibiting tumour angiogenesis in the subject, maintaining or improving one or both of the white blood cell count and red blood cell count in the subject, stimulating the immune system in the subject, increasing the production of Th1 and Th2 cytokines within a tumor in the subject, increasing the production of Th1 and Th2 cytokines within the intestine of the subject, increasing the level of Th1 and Th2 cytokines in the systemic circulation of the subject, increasing an anti-tumour immune response in the subject, increasing the responsiveness of the subject to a cancer therapy, or increasing the responsiveness of a tumour in the subject to a cancer therapy, the method comprising administration of metal ion-saturated lactoferrin or a metal ion-saturated functional variant or fragment thereof to the subject.

Another aspect of the present invention relates to a method of inhibiting tumour formation in a subject comprising administration of metal ion-saturated lactoferrin or a metal ion-saturated functional variant or fragment thereof to the subject.

Another aspect of the present invention relates to a method of inducing apoptosis in a subject in need thereof comprising administration of metal ion-saturated lactoferrin or a metal ion-saturated functional variant or fragment thereof to the subject.

Another aspect of the present invention relates to a method of inducing apoptosis of tumour cells in a subject in need thereof comprising administration of metal ion-saturated lactoferrin or a metal ion-saturated functional variant or fragment thereof to the subject.

Another aspect of the present invention relates to a method of inhibiting angiogenesis in a subject in need thereof comprising administration of metal ion-saturated lactoferrin or a metal ion-saturated functional variant or fragment thereof to the subject.

Another aspect of the present invention relates to a method of inhibiting tumour angiogenesis in a subject in need thereof comprising administration of metal ion-saturated lactoferrin or a metal ion-saturated functional variant or fragment thereof to the subject.

Another aspect of the present invention relates to a method of maintaining or improving one or both of the white blood cell count and red blood cell count of a subject comprising administration of metal ion-saturated lactoferrin or a metal ion-saturated functional variant or fragment thereof to the subject.

Another aspect of the present invention relates to a method of stimulating the immune system of a subject comprising administration of metal ion-saturated lactoferrin or a metal ion-saturated functional variant or fragment thereof to the subject.

Another aspect of the present invention relates to a method of increasing the production of Th1 and Th2 cytokines within a tumor of a subject in need thereof comprising administration of metal ion-saturated lactoferrin or a metal ion-saturated functional variant or fragment thereof to the subject.

Another aspect of the present invention relates to a method of increasing the production of Th1 and Th2 cytokines within the intestine of a subject comprising administration of metal ion-saturated lactoferrin or a metal ion-saturated functional variant or fragment thereof to the subject.

Another aspect of the present invention relates to a method of increasing the level of Th1 and Th2 cytokines in the systemic circulation of a subject comprising administration of metal ion-saturated lactoferrin or a metal ion-saturated functional variant or fragment thereof to the subject.

Another aspect of the present invention relates to a method of increasing an anti-tumour immune response in a subject comprising administration of metal ion-saturated lactoferrin or a metal ion-saturated functional variant or fragment thereof to the subject.

Another aspect of the present invention relates to a method of increasing the responsiveness of a subject to a cancer therapy comprising administration of metal ion-saturated lactoferrin or a metal ion-saturated functional variant or fragment thereof to a subject in need thereof separately, simultaneously or sequentially with administration of the therapy.

Another aspect of the present invention relates to a method of increasing the sensitivity of a tumour in a subject to a cancer therapy comprising administration of metal ion-saturated lactoferrin or a metal ion-saturated functional variant or fragment thereof to a subject in need thereof separately, simultaneously or sequentially with administration of the therapy.

Another aspect of the present invention relates to a method of inhibiting tumour growth comprising parenteral administration of a metal ion-saturated lactoferrin or a metal ion-saturated functional variant or fragment thereof to a subject in need thereof.

Another aspect of the present invention relates to a method of treating or preventing cancer comprising parenteral administration of metal ion-saturated lactoferrin or a metal ion-saturated functional variant or fragment thereof to a subject in need thereof.

Another aspect of the present invention relates to a method of inhibiting tumour growth in a subject in need thereof comprising
(a) administration of a metal ion-saturated lactoferrin or a metal ion-saturated functional variant or fragment thereof, and
(b) separate, simultaneous or sequential administration of at least one anti-tumour agent or anti-tumour therapy.

Another aspect of the present invention relates to a method of treating or preventing cancer in a subject in need thereof comprising
(a) administration of a metal ion-saturated lactoferrin or a metal ion-saturated functional variant or fragment thereof, and
(b) separate, simultaneous or sequential administration of at least one anti-tumour agent or anti-tumour therapy.

Another aspect of the present invention relates to a use of metal ion-saturated lactoferrin or a metal ion-saturated functional variant or fragment thereof in the manufacture of a composition for inhibiting tumour formation in a subject by inducing apoptosis in the subject, inducing apoptosis of tumour cells in the subject, inhibiting angiogenesis in the subject, inhibiting tumour angiogenesis in the subject, maintaining or improving one or both of the white blood cell count and red blood cell count in the subject, stimulating the immune system in the subject, increasing the production of Th1 and Th2 cytokines within a tumor in the subject, increasing the production of Th1 and Th2 cytokines within the intestine of the subject, increasing the level of Th1 and Th2 cytokines in the systemic circulation of the subject, increasing an anti-tumour immune response in the subject, increasing the responsiveness of the subject to a cancer therapy, or increasing the responsiveness of a tumour in the subject to a cancer therapy.

Another aspect of the present invention relates to a use of metal ion-saturated lactoferrin or a metal ion-saturated functional variant or fragment thereof in the manufacture of a composition for inhibiting tumour formation.

Another aspect of the present invention relates to a use of metal ion-saturated lactoferrin or a metal ion-saturated functional variant or fragment thereof in the manufacture of a composition for inducing apoptosis.

Another aspect of the present invention relates to a use of metal ion-saturated lactoferrin or a metal ion-saturated functional variant or fragment thereof in the manufacture of a composition for inducing apoptosis of tumour cells.

Another aspect of the present invention relates to a use of metal ion-saturated lactoferrin or a metal ion-saturated functional variant or fragment thereof in the manufacture of a composition for inhibiting angiogenesis.

Another aspect of the present invention relates to a use of metal ion-saturated lactoferrin or a metal ion-saturated functional variant or fragment thereof in the manufacture of a composition for inhibiting tumour angiogenesis.

Another aspect of the present invention relates to a use of metal ion-saturated lactoferrin or a metal ion-saturated functional variant or fragment thereof in the manufacture of a composition for maintaining or improving one or both of the white blood cell count and red blood cell count of a subject.

Another aspect of the present invention relates to a use of metal ion-saturated lactoferrin or a metal ion-saturated functional variant or fragment thereof in the manufacture of a composition for stimulating the immune system of a subject.

Another aspect of the present invention relates to a use of metal ion-saturated lactoferrin or a metal ion-saturated functional variant or fragment thereof in the manufacture of a composition for increasing the production of Th1 and Th2 cytokines within a tumor of a subject.

Another aspect of the present invention relates to a use of metal ion-saturated lactoferrin or a metal ion-saturated functional variant or fragment thereof in the manufacture of a composition for increasing the production of Th1 and Th2 cytokines within the intestine of a subject.

Another aspect of the present invention relates to a use of metal ion-saturated lactoferrin or a metal ion-saturated functional variant or fragment thereof in the manufacture of a composition for increasing the level of Th1 and Th2 cytokines in the systemic circulation of a subject.

Another aspect of the present invention relates to a use of metal ion-saturated lactoferrin or a metal ion-saturated functional variant or fragment thereof in the manufacture of a composition for increasing an anti-tumour immune response in a subject.

Another aspect of the present invention relates to a use of metal ion-saturated lactoferrin or a metal ion-saturated functional variant or fragment thereof in the manufacture of a composition for increasing the responsiveness of a subject to a cancer therapy.

Another aspect of the present invention relates to a use of metal ion-saturated lactoferrin or a metal ion-saturated functional variant or fragment thereof in the manufacture of a composition for increasing the sensitivity of a tumour in a subject to a cancer therapy.

Another aspect of the present invention relates to a use of metal ion-saturated lactoferrin or a metal ion-saturated functional variant or fragment thereof in the manufacture of a composition for parenteral administration for inhibiting tumour growth.

Another aspect of the present invention relates to a use of metal ion-saturated lactoferrin or a metal ion-saturated functional variant or fragment thereof in the manufacture of a composition for parenteral administration for treating or preventing cancer.

Another aspect of the present invention relates to a use of metal ion-saturated lactoferrin or a metal ion-saturated functional variant or fragment thereof in the manufacture of a composition for inhibiting tumour growth wherein the composition is administered separately, simultaneously or sequentially with at least one anti-tumour agent or anti-tumour therapy.

Another aspect of the present invention relates to a use of metal ion-saturated lactoferrin or a metal ion-saturated functional variant or fragment thereof and at least one anti-tumour agent or anti-tumour therapy in the manufacture of a composition for inhibiting tumour growth wherein the lactoferrin or functional variant or fragment administered separately, simultaneously or sequentially with the anti-tumour agent or anti-tumour therapy.

Another aspect of the present invention relates to a use of metal ion-saturated lactoferrin or a metal ion-saturated functional variant or fragment thereof in the manufacture of a composition for treating or preventing cancer wherein the composition is administered separately, simultaneously or sequentially with at least one anti-tumour agent or anti-tumour therapy.

Another aspect of the present invention relates to a use of metal ion-saturated lactoferrin or a metal ion-saturated functional variant or fragment thereof and at least one anti-tumour agent or anti-tumour therapy in the manufacture of a composition for treating or preventing cancer wherein the lactoferrin or functional variant or fragment thereof is administered separately, simultaneously or sequentially with the anti-tumour agent or anti-tumour therapy.

Another aspect of the present invention relates to a use of metal ion-saturated lactoferrin or a metal ion-saturated functional variant or fragment thereof in the manufacture of a composition for inhibiting tumour growth wherein the composition is formulated for administration separately, simultaneously or sequentially with at least one anti-tumour agent or anti-tumour therapy.

Another aspect of the present invention relates to a use of metal ion-saturated lactoferrin or a metal ion-saturated functional variant or fragment thereof and at least one anti-tumour agent or anti-tumour therapy in the manufacture of a composition for inhibiting tumour growth wherein the lactoferrin or functional variant or fragment is formulated for administration separately, simultaneously or sequentially with the anti-tumour agent or anti-tumour therapy.

Another aspect of the present invention relates to a use of metal ion-saturated lactoferrin or a metal ion-saturated functional variant or fragment thereof in the manufacture of a composition for treating or preventing cancer wherein the composition is formulated for administration separately, simultaneously or sequentially with at least one anti-tumour agent or anti-tumour therapy.

Another aspect of the present invention relates to a use of metal ion-saturated lactoferrin or a metal ion-saturated functional variant or fragment thereof and at least one anti-tumour agent or anti-tumour therapy in the manufacture of a composition for treating or preventing cancer wherein the lactoferrin or functional variant or fragment thereof is formulated for administration separately, simultaneously or sequentially with the anti-tumour agent or anti-tumour therapy.

Another aspect of the present invention relates to a parenteral unit dosage form comprising metal ion-saturated lactoferrin, a metal ion-saturated functional variant or fragment thereof or a mixture thereof and at least one anti-tumour agent.

Another aspect of the present invention relates to a dietary, nutraceutical or oral pharmaceutical composition consisting essentially of metal ion-saturated lactoferrin, a metal ion-saturated functional variant or fragment thereof or a mixture thereof and casein.

The following embodiments may relate to any of the above aspects.

In one embodiment the administration is oral, topical or parenteral administration.

In one embodiment the subject is suffering from or is susceptible to cancer.

In one embodiment the subject has suffered acute haemorrhage, is suffering from haemolytic anemia, has recently undergone strenuous exercise, or is undergoing strenuous exercise, therapy for cancer, chemotherapy, radiation therapy, surgery, immunotherapy, or treatment with a cytotoxic agent.

In one embodiment the subject has a tumour refractory to monotherapy with a chemotherapeutic, anti-angiogenic or immunotherapeutic agent. In one embodiment the subject has previously undergone unsuccessful monotherapy with a chemotherapeutic, anti-angiogenic or immunotherapeutic agent.

In one embodiment a method of the invention further comprises separate, simultaneous or sequential administration of at least one anti-tumour agent or anti-tumour therapy.

In one embodiment the metal ion-saturated lactoferrin or a metal ion-saturated functional variant or fragment thereof is administered separately, simultaneously or sequentially with at least one anti-tumour agent or anti-tumour therapy.

In one embodiment the metal ion is an ion selected from the group comprising aluminium, calcium, copper, chromium, cobalt, gold, iron, manganese, magnesium, platinum, ruthenium, selenium and zinc ions. Preferably the metal ion is an iron ion.

In one embodiment the lactoferrin is any mammalian lactoferrin including but not limited to sheep, goat, pig, mouse, water buffalo, camel, yak, horse, donkey, llama, bovine or human lactoferrin. Preferably the lactoferrin is bovine lactoferrin.

In one embodiment the metal ion-saturated lactoferrin or a metal ion-saturated functional variant or fragment thereof is at least about 25, 30, 35, 40, 45, 50, 55, 60, 65, 70, 75, 80, 85, 90, 91, 92, 93, 94, 95, 96, 97, 98, 99, 99.5 or 100% metal ion-saturated.

In one embodiment the metal ion-saturated lactoferrin or a metal ion-saturated functional variant or fragment thereof is at least about 105, 110, 115, 120, 125, 130, 135, 140, 145, 150, 155, 160, 165, 170, 175, 180, 185, 190, 195 or 200% metal ion-saturated.

In one embodiment the method comprises administration of a mixture of metal ion-saturated lactoferrin and at least one metal ion-saturated functional variant or fragment thereof.

In one embodiment the metal ion-saturated lactoferrin or a metal ion-saturated functional variant or fragment thereof and the at least one anti-tumour agent or anti-tumour therapy provide a synergistic therapeutic effect that is greater than the additive effects of either one alone. For example, there is a greater effect on inhibition of tumour formation or growth, tumour regression, cytolytic effects, immune enhancement, generation of Th1 and Th2 cytokines, or the responsiveness of a subject or a tumour to the treatment method.

In one embodiment the cancer therapy is an anti-tumour agent or anti-tumour therapy.

In one embodiment the anti-tumour therapy is selected from therapies such as, but not limited to, surgery, chemotherapies, radiation therapies, hormonal therapies, biological therapies/immunotherapies, cellular therapies, anti-angiogenic therapies, cytotoxic therapies, vaccines, nucleic acid-based vaccines (eg nucleic acids expressing a cancer antigen such as DNA vaccines including p185 vaccines), viral-based therapies (eg adeno-associated virus, lentivirus), gene therapies, small molecule inhibitor therapies, nucleotide-based therapies (eg RNAi, antisense, ribozymes etc), antibody-based therapies, oxygen and ozone treatments, embolization, and/or chemoembolization therapies.

In one embodiment the anti-tumour agent is a chemotherapeutic agent or an immunotherapeutic agent. In one embodiment the at least one anti-tumour agent is a chemotherapeutic agent. Preferably the chemotherapeutic agent is selected from tubulin disruptors, DNA intercalators, and mixtures thereof.

In one embodiment tubulin disruptors include but are not limited to: taxanes such as but not limited to Paclitaxel and Docetaxel, Vinca alkaloids, Discodermolide, Epothilones A and B, Desoxyepothilone, Cryptophycins, Curacin A, Combretastatin A-4-Phosphate, BMS 247550, BMS 184476, BMS 188791, LEP, RPR 109881A, EPO 906, TXD 258, ZD 6126, Vinflunine, LU 103793, Dolastatin 10, E7010, T138067 and T900607, Colchicine, Phenstatin, Chalcones, Indanocine, T138067, Oncocidin, Vincristine, Vinblastine, Vinorelbine, Vinflunine, Halichondrin B, Isohomohalichondrin B, ER-86526, Pironetin, Spongistatin 1, Spiket P, Cryptophycin 1, Dolastatin, Cematodin, Rhizoxin, Sarcodictyin, Eleutherobin, Laulilamide, VP-16 and D-24851.

In one embodiment DNA intercalators include but are not limited to: Acridines, Actinomycins, Anthracyclines, Benzothiopyranoindazoles, Pixantrone, Crisnatol, Brostallicin, CI-958, doxorubicin (adriamycin), actinomycin D, daunorubicin (daunomycin), bleomycin, idarubicin, mitoxantrone, cyclophosphamide, melphalan, mitomycin C, bizelesin, etoposide, mitoxantrone, SN-38, cis-platin, actinomycin D, amsacrine, DACA, Pyrazoloacridine, Irinotecan and topotecan.

In one embodiment the chemotherapeutic agent is paclitaxel, doxorubicin, epirubicin, fluorouracil, cyclophosphamide or methotrexate.

In one embodiment the anti-tumour agent is an immunotherapeutic agent. Preferably the immunotherapeutic agent is an expression plasmid encoding the T cell co-stimulator B7-1, a T cell co-stimulator, or a functionally related molecule, for example a soluble B7-Ig chimera.

In one embodiment the anti-tumour agent comprises immune cell therapy. Preferably the therapy is dendritic cell therapy.

In one embodiment the anti-tumour agent comprises one or more angiogenesis inhibitors.

In one embodiment the at least one anti-tumour agent is administered orally or parenterally, preferably by intravenous, intraperitoneal or intratumoural injection.

In one embodiment the metal ion-saturated lactoferrin or metal ion-saturated functional variant or fragment thereof is administered daily for at least about 1, 2, 3, 4, 5, 6, 7, 8, 9 or 10 weeks before administration of the anti-tumour agent or anti-tumour therapy.

In one embodiment the metal ion-saturated lactoferrin or a metal ion-saturated functional variant or fragment thereof is administered for at least about 1, 2, 3, 4, 5, 6, 7, 8, 9, 10, 11, 12, 13, 14, 15, 16, 17, 18, 19, 20 or 21 days or for at least about 1, 2, 3, 4, 5, 6, 7 or 8 weeks or for at least about 1, 2, 3, 4, 5 or 6 months before administration of the anti-tumour agent or the anti-tumour therapy

In one embodiment the metal ion-saturated lactoferrin or a metal ion-saturated functional variant or fragment thereof is administered for at least about 1, 2, 3, 4, 5, 6, 7, 8, 9, 10, 11, 12, 13, 14, 15, 16, 17, 18, 19, 20 or 21 days or for at least about 1, 2, 3, 4, 5, 6, 7 or 8 weeks or for at least about 1, 2, 3, 4, 5 or 6 months after administration of the anti-tumour agent or the anti-tumour therapy has begun.

In one embodiment the metal ion-saturated lactoferrin or a metal ion-saturated functional variant or fragment thereof is administered at least once daily including continuously over a day by parenteral drip for example.

In one embodiment the tumour or the cancer is a leukemia, lymphoma, multiple myeloma, a hematopoietic tumor of lymphoid lineage, a hematopoietic tumor of myeloid lineage, a colon carcinoma, a breast cancer, a melanoma, a skin cancer or a lung cancer.

In one embodiment the tumour is, the tumour cells are or the cancer is a leukemia such as but not limited to, acute leukemia, acute lymphocytic leukemia, acute granulocytic leukemia, acute myelocytic leukemia such as myeloblastic, promyelocytic, myelomonocytic, monocytic, erythroleukemia leukemia and myelodysplastic syndrome, chronic leukemia such as but not limited to, chronic myelocytic leukemia, chronic granulocytic leukemia, chronic lymphocytic leukemia, and hairy cell leukemia.

In one embodiment the tumour is, the tumour cells are or the cancer is a lymphoma such as but not limited to Hodgkin's disease and non-Hodgkin's disease.

In one embodiment the tumour is, the tumour cells are from or the cancer comprises a hematopoietic tumor of myeloid lineage such as but not limited to acute and chronic myelogenous leukemia, smoldering multiple myeloma, nonsecretory myeloma and osteosclerotic myeloma.

In one embodiment the tumour is, the tumour cells are from or the cancer comprises a hematopoietic tumor of lymphoid lineage, including leukemia, acute and chronic lymphocytic leukemia, acute and chronic lymphoblastic leukemia, B-cell lymphoma, T-cell lymphoma, Burkitts lymphoma.

In one embodiment the tumour is, the tumour cells are from or the cancer comprises a hematopoietic tumor of B lymplioid lineage, including B-Cell Chronic Lymphocytic Leukemia (B-CLL)/Small Lymphocytic Lymphoma (SLL), Lymphoplasmacytoid Lymphoma, Follicle Center Lymphoma, Follicular Small Cleaved Cell (FSC), Follicular Mixed Cell (FM), Marginal Zone B-cell Lymphoma, Hairy Cell Leukemia, Plasmacytoma/Myeloma B-Cell Prolymphocytic Leukemia (B-PLL), Mantle Cell Lymphoma, Follicle Center Lymphoma, Follicular Small Cleaved Cell (FSC), Follicle Center Lymphoma (follicular large cell), B-Cell Large B-Cell Lymphoma, Precursor B-Lymphoblastic Leukemia/Lymphoma (PB-LBL/L), Burkitt's Lymphoma, High-Grade B-Cell Lymphoma, Burkitt's-like, Small lymphocytic/pro-lymphocytic lymphoma (SLL), Follicular lymphoma (few large cells), Lymphoplasmacytoid lymphoma, Marginal zone lymphoma.

In one embodiment the tumour is, the tumour cells are from or the cancer comprises a hematopoietic tumor of T lymphoid lineage, including Large Granular Lymphocyte Leukemia, Adult T-Cell Leukemia/Lymphoma (ATL/L) [smoldering], Mycosis Fungoides/Sezary Syndrome, T-cell Chronic Lymphocytic Leukemia/Prolymphocytic Leukemia (T-CLL/PLL), Adult T-Cell Leukemia/Lymphoma (ATL/L) [chronic], Angiocentric Lymphoma, Angioimmunoblastic Lymphoma, Peripheral T-Cell Lymphomas, Intestinal T-Cell Lymphoma, Anaplastic Large Cell Lymphoma, Precursor T-lymphoblastic leukemia/lymphoma (T-LBL/L), Adult T-cell leukemia/Lymphoma (ATLL) [acute and lymphomatous], Large granular lymphocyte leukemia, Adult T-cell leukemia/lymphoma (ATL/L), Mycosis fungoides/Sezary syndrome.

In one embodiment the tumour is a large tumour. In one embodiment the tumour is or the cancer comprises
(a) a tumour that is at least about 0.3, 0.4 or 0.5 cm in diameter, or
(b) a tumour that is refractory to monotherapy with one at least one immunotherapeutic, anti-angiogenic or chemotherapeutic agent.

In one embodiment one or both of the white blood cell count and red blood cell count of the subject is maintained or improved.

In one embodiment the tumour is reduced in size or substantially eradicated.

In one embodiment the metal ion-saturated lactoferrin or a metal ion-saturated functional variant or fragment thereof is administered in a dosage form comprising digestible protein, preferably casein or other protective protein.

In one embodiment the metal ion-saturated lactoferrin or a metal ion-saturated functional variant or fragment thereof and the therapy are administered simultaneously.

In one embodiment the subject is undergoing treatment with a cytotoxic agent.

In one embodiment the composition is a food, drink, food additive, drink additive, dietary supplement, nutritional product, medical food, nutraceutical, medicament or pharmaceutical. Preferably the composition is formulated for oral or topical administration. Preferably the composition is formulated for oral or parenteral administration. In one embodiment the composition is a milk fraction.

In one embodiment the metal ion-saturated lactoferrin or a metal ion-saturated functional variant or fragment thereof is formulated for administration separately, simultaneously or sequentially with at least one anti-tumour agent or anti-tumour therapy described above.

In one embodiment the metal ion-saturated lactoferrin or a metal ion-saturated functional variant or fragment thereof is formulated for coadministration with the at least one anti-tumour agent or anti-tumour therapy described above.

In one embodiment the metal ion-saturated lactoferrin or a metal ion-saturated functional variant or fragment thereof is formulated for sequential administration with the at least one anti-tumour agent or anti-tumour therapy described above.

In one embodiment a composition of the invention or a composition employed in a method of the invention provides a population of lactoferrin polypeptides or functional variants or fragments thereof wherein at least about 25, 30, 35, 40, 45, 50, 55, 60, 65, 70, 75, 80, 85, 90, 91, 92, 93, 94, 95, 96, 97, 98, 99, 99.5 or 100% of the available metal ion-binding pockets in the population are bound to a metal ion, preferably an iron ion.

In one embodiment a composition of the invention or a composition employed in a method of the invention provides a population of lactoferrin polypeptides or functional variants or fragments thereof wherein about 100% of the available metal ion-binding pockets in the population are bound to a metal ion, preferably an iron ion, and additional metal ions are bound to the lactoferrin molecules in non-specific binding sites so that the lactoferrins 105, 110, 115, 120, 125, 130, 135, 140, 145, 150, 155, 160, 165, 170, 175, 180, 185, 190, 195 or 200% metal ion saturated on a stoichiometric basis.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7).

The entire disclosures of all applications, patents and publications, cited above and below, if any, are hereby incorporated by reference.

### BRIEF DESCRIPTION OF THE DRAWINGS

FIGS. 1A and 1B are graphs which show the effects of oral feeding of iron-saturated bovine lactoferrin (Fe-Lf) and natural bovine lactoferrin (bLf) on tumour growth and anti-tumor cytotoxic T-lymphocyte (CTL) and NK cell activity of iron-saturated bovine lactoferrin and natural bLf.

FIGS. 2A and 2B are graphs which show that inhibition of tumour growth correlates with the level of anti-tumour CTL and NK cell activity.

FIGS. 3A, 3B and 3C are graphs which show the effects of oral feeding of iron-saturated bovine lactoferrin (Fe-Lf) or natural bLf in combination with one or both of over-expression of B7-1 and inhibition of HIF-1α on tumour growth and the correlation of same with the level of anti-tumour CTL and NK cell activity.

FIGS. 4A and 4B are graphs which show the effects of oral feeding of iron-saturated bovine lactoferrin (Fe-Lf) and administration of chemotherapeutic drugs on tumour growth.

FIGS. 5A and 5B are graphs which show the effects of oral feeding of iron-saturated bovine lactoferrin (Fe-Lf) and administration of doxorubicin and paclitaxel either alone or in combination with one another on tumour cell apoptosis and the correlation of same with the level of anti-tumour CTL and NK cell activity.

FIG. 6 comprises a photograph of a gel and a corresponding graph which shows the detection of iron-saturated bovine lactoferrin (Fe-Lf) in the systemic circulation.

FIG. 7 comprises a photograph of a gel and a corresponding graph which shows detection of iron-saturated bovine lactoferrin at the tumour site and in the small intestine.

FIG. 8A comprises a photograph of a gel and a corresponding graph which shows detection of iron-saturated bovine lactoferrin (Fe-Lf) in multiple organs of mice fed an iron-saturated bovine lactoferrin diet.

FIG. 8B comprises a photograph of a gel which shows detection of endogenous lactoferrin in multiple organs of mice fed the AIN93G control diet.

FIG. 9 comprises two graphs which show the effects of oral feeding of iron-saturated bovine lactoferrin (Fe-Lf) and intravenous injection of fused DC-EL-4 hybrid cells on tumour growth.

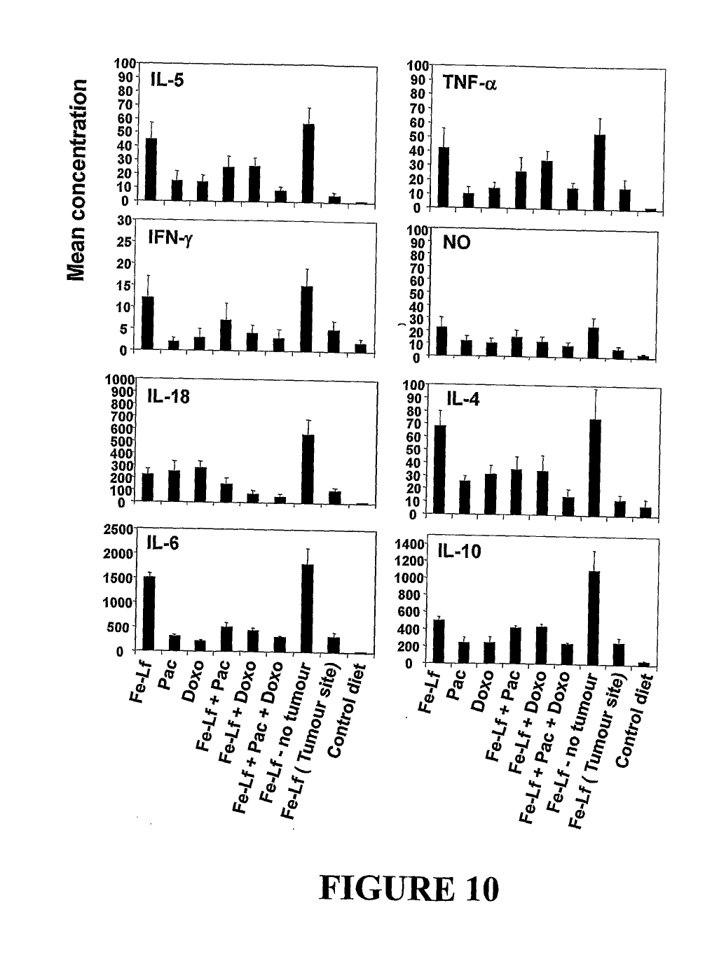

FIG. 10 comprises eight graphs which show the effects of oral feeding of iron-saturated bovine lactoferrin (Fe-Lf), paclitaxel and doxorubicin alone or in various combinations on levels of Th1 and Th2 cytokines in the intestines and tumours of mice.

FIG. 11 is two graphs showing that the anti-tumour activity of bovine lactoferrin (Lf) and sensitization of tumours to chemotherapy depends on the level of Fe-saturation. (A) Mice were fed the control AIN93G diet, and the same diet supplemented with either fully Fe-saturated Lf, 50% Fe-saturated Lf, native Lf, or apoLf. Day 0 refers to the day the mice were placed on their diets. After 2 weeks on the diets, EL-4 cells were injected into the flanks of mice. Paclitaxel (30 mg/Kg) was administered as indicated tumour size monitored for 77 days, or until tumours reached 1 cm in diameter. Each point represents the mean tumour size with 95% confidence intervals for either 10 mice, or the number of mice indicated. (B) Effects on anti-tumor CTL activity. Splenocytes were harvested from mice in FIG. 1A at day 77 (or day 56 in the case of controls) and tested for their cytolytic activity against EL-4 target cells. The percent cytotoxicity is plotted against various effector-to-target cell ratios (E:T ratios). Each point represents the mean percent cytotoxicity obtained from 5 mice. Error bar represents 95% confidence intervals.

FIG. 12 is two graphs showing the dose-response of Fe-saturated Lf. (A) Mice were fed the control diet, and the same diet supplemented with different levels of Fe-saturated Lf ranging from 0, 1, 5, 25, and 100 g per 2.4 Kg of diet. Day 0 refers to the day the mice were placed on their diets. After 2 weeks on the diets, EL-4 cells were injected into the flanks of mice. Paclitaxel (30 mg/Kg) was administered as indicated and tumour size was monitored for 77 days, or until tumours reached 1 cm in diameter. Each point represents the mean tumour size with 95% confidence intervals for either 10 mice, or the number of mice indicated. The numbers of mice from each group which completely rejected the tumour challenge is shown above the x-axis. (B) Effects on anti-tumor CTL activity. Splenocytes were harvested from mice in FIG. 2A at day 77 (or day 56 in the case of controls) and tested for their cytolytic activity against EL-4 target cells. The percent cytotoxicity is plotted against various effector-to-target cell ratios (E:T ratios). Each point represents the mean percent cytotoxicity obtained from 5 mice. Error bar represents 95% confidence intervals.

FIG. 13 is a graph showing the detection of anti-tumour factors released into the systemic circulation in response to Fe-saturated Lf. Sera collected after 6 weeks of feeding mice Fe-saturated Lf or the control AIN-93 diet was tested for its ability to trigger the apoptosis of cultured EL-4 tumour cells. The apoptotic index was (AI) was calculated after measuring the numbers of apoptotic cells following staining with TUNEL, annexin-V-fluos, and trypan blue. The AI of cultured EL-4 cells was included as a control for spontaneous apoptosis.

FIG. 14 is four graphs showing that 100% Fe-saturated Lf increases the sensitivity of different tumour types to different chemotherapeutic agents. Tumours were established in mice fed the control AIN93G diet, or the same diet supplemented with iron-saturated bovine Lf, as described previously. (A) Effects of epirubucin. Mice were randomized into 3 groups: an untreated control group fed the control diet, a group fed the control diet receiving epirubucin, and a group fed Lf receiving epirubucin. Epirubucin (15 mg/Kg) was administered as indicated. (B) Effects on anti-tumor CTL and NK cell activity. Splenocytes were harvested from mice in FIG. 4A and tested for their cytolytic activity against EL-4 and LLC target cells. The percent cytotoxicity is plotted against various effector-to-target cell ratios (E:T ratios). Each point represents the mean percent cytotoxicity obtained from 5 mice. Bar represents 95% confidence intervals. (C) Effects of fluorouracil. Mice were randomized into an untreated control group fed the control diet, a group fed the control diet receiving fluorouracil, and a group fed Lf receiving fluorouracil. Fluorouracil (150 mg/Kg) was administered as indicated. (D) Effects on anti-tumor CTL and NK cell activity. Splenocytes were harvested from mice in FIG. 4C and tested for their cytolytic activity against B16 and EL-4 target cells. The percent cytotoxicity is plotted against various effector-to-target cell ratios (E:T ratios). Each point represents the mean percent cytotoxicity obtained from 5 mice. Bar represents 95% confidence intervals. (E) Effects of cyclophosphamide and methotrexate. Mice were randomized into an untreated control group fed the control diet, groups fed the control diet receiving either cyclophosphamide or methotrexate, and groups fed Lf receiving either cyclophosphamide or methotrexate. Cyclophosphamide (100 mg/Kg) and methotrexate (30 mg/Kg) were administered as indicated. (F) Effects on anti-tumor CTL and NK cell activity. Splenocytes were harvested from mice in FIG. 4E and tested for their cytolytic activity against EL-4 target cells. The percent cytotoxicity is plotted against various effector-to-target cell ratios (E:T ratios). Each point represents the mean percent cytotoxicity obtained from 5 mice. Bar represents 95% confidence intervals.

FIG. 15 is a graph showing that Fe-saturated Lf is inherently more active than natural Lf in its ability to stimulate intestinal cytokine production. Fe-saturated Lf, natural Lf, and bovine serum albumin as a control were incubated in intestinal loops, and IL-18 released by the intestine (secreted) and present in the supernatant of intestinal homogenates (lysates) was measured. Intestinal loops were also incubated in the absence of a stimulatory protein to measure the natural levels of IL-18.

### DETAILED DESCRIPTION OF THE INVENTION

### 1. Definitions

The term “anti-tumour factors” refers at least to apoptosis inducing factors and may include anti-tumour cytolytic antibodies and tumoricidal cytokines such as TNF-α.

The term “anti-tumour immune response” refers to the ability of metal ion-saturated lactoferrin to stimulate the generation of antigen-specific cytolytic activity (the activity of immune cells, particularly cytotoxic T-lymphocytes) and/or NK cell activity, improve the cellular immune response to antigens (through the activity of at least cytotoxic T-lymphocytes), improve immune protection (by at least restoring the activity of cytotoxic T-lymphocytes and/or NK cells and enhancing cytokine production), restore immune protection (by at least restoring or stimulating the activity of cytotoxic T-lymphocytes and/or NK cell activity and enhancing cytokine production), generate pro-inflammatory and immunoregulatory mediators (Th1 and Th2 cytokines), and/or generate anti-tumour cytolytic antibodies and tumoricidal cytokines such as TNF-α.

The term “comprising” as used in this specification and the claims means “consisting at least in part of”. When interpreting statements in this specification and the claims that include that term, the features, prefaced by that term in each statement, all need to be present but other features can also be present.

An “effective amount” is the amount required to confer therapeutic effect. The interrelationship of dosages for animals and humans (based on milligrams per meter squared of body surface) is described by Freireich, et al. (1966). Body surface area can be approximately determined from height and weight of the subject. See, e.g., Scientific Tables, Geigy Pharmaceuticals, Ardley, N.Y., 1970, 537. Effective doses also vary, as recognized by those skilled in the art, dependent on route of administration, excipient usage, and the like.

The terms “enhance the immune system” and “stimulate the immune system” (and different tenses of these terms) refer to the ability of metal ion-saturated lactoferrin to stimulate the generation of antigen-specific cytolytic activity (the activity of immune cells, particularly cytotoxic T-lymphocytes) and/or NK cell activity, improve the cellular immune response to antigens (through the activity of at least cytotoxic T-lymphocytes), improve immune protection (by at least restoring the activity of cytotoxic T-lymphocytes and/or NK cells and enhancing cytokine production), restore immune protection (by at least restoring or stimulating the activity of cytotoxic T-lymphocytes and/or NK cell activity and enhancing cytokine production) or generate pro-inflammatory and immunoregulatory mediators (Th1 and Th2 cytokines).

The term “functional fragment” is intended to mean a naturally occurring or non-naturally occurring portion of a lactoferrin polypeptide that has one or two metal ion binding pockets and that has activity when assayed according the examples below. Useful lactoferrin fragments include truncated lactoferrin polypeptides (including but not limited to SEQ ID NO. 11), metal ion-binding hydrolysates of lactoferrin, fragments that comprise the N-lobe binding pocket (including but not limited to N-lobe sequences SEQ ID NO.s 5 to 10), fragments that comprise the C-lobe binding pocket (including but not limited to C-lobe sequences SEQ ID NO.s 12 to 17), and metal ion-binding fragments generated (by artificial or natural processes) and identified by known techniques as discussed below.

The term “functional variant” is intended to mean a variant of a lactoferrin polypeptide that has activity when assayed according the examples below and so is able to inhibit tumour formation or inhibit tumour growth.

The term “glycosylated” when used in relation to a lactoferrin polypeptide, functional variant or fragment is intended to mean that the lactoferrin is fully or partially glycosylated with naturally occurring or non-naturally occurring human or bovine glycosyl groups. Glycoslyated and aglycosyl forms of lactoferrin are known (see Pierce, et al. (1991); Metz-Boutigue, et al. (1984); van Veen, et al. (2004)).

The term “increasing the responsiveness of a subject” is intended to mean that a subject exhibits a greater reduction in the rate of tumour growth, in tumour size, or in clinical symptoms of disease than a subject who is not subjected to a method of the invention.

The term “increasing the sensitivity of a tumour” is intended to mean that a tumour exhibits a greater reduction in the rate of tumour growth, in tumour size, or is eradicated whereas a tumour that is not subjected to a method of the invention will not exhibit these effects.

The term “immunotherapeutic agent” is intended to mean an agent that stimulates anti-tumour immunity. Agents that stimulate anti-tumour activity are preferably those that directly or indirectly stimulate T-cells and/or NK cells to kill tumour cells. An in vitro assay for assessing whether a selected agent stimulates anti-tumour immunity is the CTL assay described below.

The term “inhibiting tumour formation” is intended to mean that tumours do not form, or that tumours form but do not establish or grow, or that tumours form but remain small, benign and do not become cancerous or metastasize, or that tumours grow more slowly. Tumour formation may be monitored through CT scans and tumor markers where available.

The term “inhibiting tumour growth” is intended to mean that tumours do not form in a subject treated according to the invention, or that one or more tumours that may be present in a subject treated according to the invention do not grow in size or become cancerous or metastasize, or that one or more tumours present in a subject treated according to the invention reduce in size (preferably by at least about 20, 30, 40, 50, 60, 70, 80, 90 or 100% by volume) or that one or more tumours present in a subject treated according to the invention are eradicated. Tumour size may be monitored through CT scans and tumor markers where available.

The terms “iron-lactoferrin” and “iron-saturated lactoferrin” as used herein are intended to refer to a population of lactoferrin polypeptides providing a population of iron-binding pockets where at least about 25, 30, 35, 40, 45, 50, 55, 60, 65, 70, 75, 80, 85, 90, 91, 92, 93, 94, 95, 96, 97, 98, 99, 99.5, 99.9 or 100% of the metal ion-binding pockets present in the population have an iron ion bound.

The term “lactoferrin polypeptide” refers to a non-glycosylated or glycosylated wild-type lactoferrin amino acid sequence (including but not limited to SEQ ID NO.s 1 to 4) or homologous lactoferrin sequences from other species such as those described below. A lactoferrin polypeptide has two metal-ion binding pockets and so can bind metal ions in a stoichiometric ratio of 2 metal ions per lactoferrin molecule. One metal ion-binding pocket is present in the N-terminal lobe (N-lobe) of lactoferrin and the other pocket is present in the C-terminal lobe (C-lobe) (Moore et al, 1997). Verified sequences of bovine and human lactotransferrins (lactoferrin precursors), lactoferrins and peptides therein can be found in Swiss-Prot (http://au.expasy.org/cgi-bin/sprot-search-fil). Indicative lactoferrin polypeptides include the bovine lactotransferrin precursor accession number P24627 (SEQ ID NO. 1), bovine lactoferrin (SEQ ID NO. 2), the human lactotransferrin precursor accession number P02788 (SEQ ID NO. 3) and human lactoferrin (SEQ ID NO. 4).

The term “large tumour” is intended to mean a tumour that is refractory to monotherapy with one at least one immunotherapeutic, anti-angiogenic or chemotherapeutic agent, preferably refractory to monotherapy with at least one at least one immunotherapeutic or chemotherapeutic agent. In one embodiment a large tumour is a tumour that is at least about 0.3, 0.4, 0.5, 0.6, 0.7 or 0.8 cm in diameter. In one embodiment a large tumour is a tumour that is about 0.3 to about 0.8, about 0.4 to about 0.8, about 0.5 to about 0.8, about 0.6 to about 0.8 or about 0.7 to about 0.8 cm in diameter. In one embodiment a large tumour is a tumour that is refractory to monotherapy by immunotherapy or anti-angiogenic therapy or chemotherapy.

The term “metal ion-binding” is intended to refer to binding of a metal ion in an iron binding pocket of a lactoferrin polypeptide or in an iron binding pocket of a fragment of a lactoferrin polypeptide that is still able to form the iron binding pocket.

The term “metal ion-saturated lactoferrin” is intended to refer to a population of lactoferrin polypeptides that provides a population of metal ion-binding pockets where at least about 25% of the metal ion-binding pockets present in the population have a metal ion bound. It should be understood that the population may contain polypeptides of different species; for example, some molecules binding no ion and others each binding one or two ions. In cases where different metal ions are used, some molecules may bind an iron ion and others a different ion.

Equally, the term “metal ion-saturated lactoferrin fragment” is intended to refer to a population of lactoferrin polypeptide fragments that provides a population of metal ion-binding pockets where at least about 25% of the metal ion-binding pockets present in the population have a metal ion bound.

The present invention may employ a mixture of lactoferrin polypeptides and lactoferrin fragments. In such an embodiment, the population of metal ion-binding pockets is made up of two pockets for every lactoferrin polypeptide and one or two pockets for every lactoferrin fragment, depending on the nature of the fragments.

The degree of saturation may determined by spectrophotometric analysis (Brock & Azabe, 1976; Bates et al, 1967; Bates et al, 1973). It should be understood that there may be metal ion-exchange between lactoferrin polypeptides. In one embodiment, iron saturated lactoferrin may be prepared by the method of Law, et al (1977). In another embodiment, iron saturated lactoferrin may be prepared by the method of Kawakami et al (1993). Metal-ion saturated lactoferrin may be prepared by binding metal ions to the metal ion binding sites in lactoferrin, including the metal ion binding pockets such as the Fe binding pockets and other non-specific binding sites on the lactoferrin molecule or lactoferrin fragment.

In one embodiment at least about 25, 30, 35, 40, 45, 50, 55, 60, 65, 70, 75, 80, 85, 90, 91, 92, 93, 94, 95, 96, 97, 98, 99, 99.5, 99.9 or 100% of the metal ion-binding pockets present in the population of lactoferrin molecules have a metal ion bound and useful ranges may be selected between any of the foregoing values (for example, about 25 to about 100%, about 30 to about 100%, about 35 to about 100%, about 40 to about 100%, about 45 to about 100%, about 50 to about 100%, about 55 to about 100%, about 60 to about 100%, about 65 to about 100%, about 70 to about 100%, about 75 to about 100%, about 80 to about 100%, about 85 to about 100%, about 90 to about 100%, about 95 to about 100% and about 99 to about 100%). In one embodiment the metal ion-saturated lactoferrin is super-saturated lactoferrin.

The term “oral administration” includes oral, buccal, enteral and intra-gastric administration.

The term “parenteral administration” includes but is not limited to topical (including administration to any dermal, epidermal or mucosal surface), subcutaneous, intravenous, intraperitoneal, intramuscular and intratumoural (including any direct administration to a tumour) administration.

The term “pharmaceutically acceptable carrier” is intended to refer to a carrier including but not limited to an excipient, diluent or auxiliary that can be administered to a subject as a component of a composition of the invention. Preferred carriers do not reduce the activity of the composition and are not toxic when administered in doses sufficient to deliver an effective amount of a lactoferrin polypeptide or functional variant or fragment thereof. The formulations can be administered orally, nasally or parenterally.

The term “subject” is intended to refer to an animal, preferably a mammal, more preferably a mammalian companion animal or human. Preferred companion animals include cats, dogs and horses.

The term “super-saturated lactoferrin” refers to a population of lactoferrin polypeptides or functional fragments providing a population of metal ion-binding pockets where sufficient metal ions are available to fill 100% of the binding pockets and additional metal ions are present and bound by non-specific binding sites on the lactoferrin polypeptide or lactoferrin fragment. In other words, a stoichiometric excess of metal ions is provided. Preferably no free metal ions are present in a composition of the invention comprising super-saturated lactoferrin, although metal ion exchange between binding pockets, between non-specific binding sites and between binding pockets and non-specific binding sites may occur. Preferably super-saturated lactoferrin does not form insoluble aggregates. In one embodiment the super-saturated lactoferrin is at least about 105, 110, 115, 120, 125, 130, 135, 140, 145, 150, 155, 160, 165, 170, 175, 180, 185, 190, 195 or 200% metal ion saturated, preferably iron saturated.

The term “treat” and its derivatives should be interpreted in their broadest possible context. The term should not be taken to imply that a subject is treated until total recovery. Accordingly, “treat” broadly includes amelioration and/or prevention of the onset of the symptoms or severity of a particular condition. The term “treat” also broadly includes the maintenance of good health for sensitive individuals and building stamina for disease prevention.

The term “variant” refers to a naturally occurring (an allelic variant, for example) or non-naturally occurring (an artificially generated mutant, for example) lactoferrin polypeptide or lactoferrin fragment that varies from the predominant wild-type amino acid sequence of a lactoferrin polypeptide of a given species (such as those listed below) or fragment thereof by the addition, deletion or substitution of one or more amino acids.

Generally, polypeptide sequence variant possesses qualitative biological activity in common when assayed according to the examples below. Further, these polypeptide sequence variants may share at least about 50%, 55%, 60%, 65%, 70%, 75%, 80%, 85%, 90%, 95%, 96%, 97%, 98% or 99% sequence identity. Also included within the meaning of the term “variant” are homologues of lactoferrin polypeptides. A homologue is typically a polypeptide from a different species but sharing substantially the same biological function or activity as the corresponding polypeptide disclosed herein.

Preferred variant polypeptides preferably have at least about 70, 75, 80, 85, 90, 95 or 99% identity, preferably at least about 90, 95 or 99% identity to a sequence selected from SEQ ID NO.s 1 to 4. Variant fragments preferably have at least about 70, 75, 80, 85, 90, 95 or 99% identity, preferably at least about 90, 95 or 99% identity to a fragment described herein, including but not limited to SEQ ID NO.s 5 to 17. Identity can be determined by comparing a candidate amino acid sequence to a sequence described herein, such as a lactoferrin polypeptide or fragment thereof using BLASTN (from the BLAST suite of programs, version 2.2.5 [November 2002]) in b12seq (Tatusova, et al. (1999)) that is publicly available from NCBI (ftp://ftp.ncbi.nih.gov/blast/). The default parameters of b12seq may be utilized.

Conservative substitutions of one or several amino acids of a lactoferrin polypeptide sequence without significantly altering its biological activity are also useful. A skilled artisan will be aware of methods for making phenotypically silent amino acid substitutions (see for example Bowie et al., (1990)).

### 2. Lactoferrin Polypeptides

In addition to the useful lactoferrin polypeptides and fragments listed above, examples of lactoferrin amino acid and mRNA sequences that have been reported and are useful in methods of the invention include but are not limited to the amino acid (Accession Number NP_{—}002334) and mRNA (Accession Number NM_{—}002343) sequences of human lactoferrin; the amino acid (Accession Numbers NP_{—}851341 and CAA38572) and mRNA (Accession Numbers X54801 and NM_{—}180998) sequences of bovine lactoferrin; the amino acid (Accession Numbers JC2323, CAA55517 and AAA97958) and mRNA (Accession Number U53857) sequences of goat lactoferrin; the amino acid (Accession Number CAA09407) and mRNA (Accession Number AJ010930) sequences of horse lactoferrin; the amino acid (Accession Numbers NP_{—}999527, AAL40161 and AAP70487) and mRNA (Accession Number NM_{—}214362) sequences of pig lactoferrin; the amino acid (Accession Number NP_{—}032548) and mRNA (Accession Number NM_{—}008522) sequences of mouse lactoferrin; the amino acid (Accession Number CAA06441) and mRNA (Accession Number AJ005203) sequences of water buffalo lactoferrin; and the amino acid (Accession Number CAB53387) and mRNA (Accession Number AJ131674) sequences of camel lactoferrin. These sequences may be used according to the invention in wild type or variant form. Polypeptides encoded by these sequences may be isolated from a natural source, produced as recombinant proteins or produced by organic synthesis, using known techniques.

Methods for generating useful polypeptides and variants are known in the art and discussed below. Useful recombinant lactoferrin polypeptides and fragments and methods of producing them are reported in US patent specifications U.S. Pat. No. 5,571,691, U.S. Pat. No. 5,571,697, U.S. Pat. No. 5,571,896, U.S. Pat. No. 5,766,939, U.S. Pat. No. 5,849,881, U.S. Pat. No. 5,849,885, U.S. Pat. No. 5,861,491, U.S. Pat. No. 5,919,913, U.S. Pat. No. 5,955,316, U.S. Pat. No. 6,066,469, U.S. Pat. No. 6,080,599, U.S. Pat. No. 6,100,054, U.S. Pat. No. 6,111,081, U.S. Pat. No. 6,228,614, U.S. Pat. No. 6,277,817, U.S. Pat. No. 6,333,311, U.S. Pat. No. 6,455,687, U.S. Pat. No. 6,569,831, U.S. Pat. No. 6,635,447, US 2005-0064546 and US 2005-0114911.

Useful variants also include bovine lactoferrin variants bLf-a and bLf-b (Tsuji, et al. (1989); Yoshida, et al. (1991)). Further useful variants include glycoslyated and aglycosyl forms of lactoferrin (Pierce, et al. (1991); Metz-Boutigue, et al. (1984); van Veen, et al. (2004)) and glycosylation mutants (having variant points of glycosylation or variant glycosyl side chains).

Useful fragments include the N-lobe and C-lobe fragments (Baker, et al., 2002) and any other lactoferrin polypeptides that retain a lactoferrin binding pocket, such as truncated lactoferrin polypeptides.

Useful truncated lactoferrin polypeptides include polypeptides of SEQ ID NO.s 1, 2, 3 or 4 truncated by about 1 to about 300 amino acids, preferably about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300 amino acids or more, and including polypeptides truncated at the N-terminus, at the C-terminus or at both the N-terminus and C-terminus, provided that the truncated polypeptide retains at least one of the N-lobe or the C-lobe metal ion-binding pockets. SEQ ID NO.s 5 to 10 are examples of C-terminal truncations retaining the N-lobe metal ion-binding pocket. SEQ ID NO.s 11 to 15 and 17 are examples of N-terminal truncations retaining the C-lobe metal ion-binding pocket. SEQ ID NO. 16 is an example of a sequence truncated at both ends retaining the C-lobe metal ion-biding pocket. It is reported that residues Asp 60, Tyr 92, Tyr 192, His 253 of SEQ ID NO. 2 are the amino acid metal ion ligands in the N-lobe. It is reported that residues Asp 395, Tyr 433, Tyr 526, His 595 of SEQ ID NO. 2 are the amino acid metal ion ligands in the C-lobe. (Karthikeyan, et al., 1999)

Candidate variants or fragments of lactoferrin for use according to the present invention may be generated by techniques including but not limited to techniques for mutating wild type proteins (see Sambrook, et al. (1989) and elsewhere of a discussion of such techniques) such as but not limited to site-directed mutagenesis of wild type lactoferrin and expression of the resulting polynucleotides; techniques for generating expressible polynucleotide fragments such as PCR using a pool of random or selected primers; techniques for full or partial proteolysis or hydrolysis of wild type or variant lactoferrin polypeptides; and techniques for chemical synthesis of polypeptides. Variants or fragments of lactoferrin may be prepared by expression as recombinant molecules from lactoferrin DNA or RNA, or variants or fragments thereof. Nucleic acid sequences encoding variants or fragments of lactoferrin may be inserted into a suitable vector for expression in a cell, including eukaryotic cells such as but not limited to Aspergillus or bacterial cells such as but not limited to E. coli. Lactoferrin variants or fragments may be prepared using known PCR techniques including but not limited to error-prone PCR and DNA shuffling. Error-prone PCR is a process for performing PCR under conditions where the copying fidelity of the DNA polymerase is low, such that a high rate of point mutations is obtained along the entire length of the PCR product (Leung, et al. (1989); Cadwell, et al. (1992)). DNA shuffling refers to forced homologous recombination between DNA molecules of different but highly related DNA sequence in vitro, caused by random fragmentation of the DNA molecule based on sequence homology, followed by fixation of the crossover by primer extension in a PCR reaction (Stemmer (1994)). Suitable lactoferrin nucleic acid sequences for use in such methods may be generated by known methods including, for example, reverse transcription-PCR(RT-PCR) of tissue RNA isolates. Suitable primers for RT-PCR may be designed with reference to the mRNA sequences listed above. Commercial kits are available for RT-PCR (for example, Cells-to-cDNA™ kits from Ambion, USA).

Variants or fragments of lactoferrin may also be generated by known synthetic methods (see Kimmerlin, et al., 2005, for example).

Metal ion-binding variants or fragments of lactoferrin may be obtained by known techniques for isolating metal-binding polypeptides including but not limited to metal affinity chromatography, for example. Candidate variants or fragments of lactoferrin may be contacted with free or immobilised metal ions, such as Fe³⁺ and purified in a suitable fashion. For example, candidate variants or fragments may be contacted at neutral pH with a metal ion immobilised by chelation to a chromatography matrix comprising iminodiacetic acid or tris(carboxymethyl)ethylenediamine ligands. Bound variants or fragments may be eluted from the supporting matrix and collected by reducing the pH and ionic strength of the buffer employed. Metal-bound variants or fragments may be prepared according to the methods described above and below and described in the Examples below.

Functional variants, fragments and hydrolysates of lactoferrin may be obtained by selecting variants, fragments and hydrolysates of lactoferrin and assessing their efficacy in methods of the present invention by employing the methodologies set out in the Examples described below.

In one embodiment the lactoferrin is any mammalian lactoferrin including but not limited to sheep, goat, pig, mouse, water buffalo, camel, yak, horse, donkey, llama, bovine or human lactoferrin. Preferably the lactoferrin is bovine lactoferrin.

In another embodiment the lactoferrin is any recombinant mammalian lactoferrin including but not limited to recombinant sheep, goat, pig, mouse, water buffalo, camel, yak, horse, donkey, llama, bovine or human lactoferrin. Preferably the lactoferrin is recombinant bovine lactoferrin. Recombinant lactoferrin may be produced by expression in cell free expression systems or in transgenic animals, plants, fungi or bacteria, or other useful species. Alternatively, lactoferrin may be produced using known organic synthetic methods.

In yet another embodiment the lactoferrin is isolated from milk, preferably sheep, goat, pig, mouse, water buffalo, camel, yak, horse, donkey, llama, bovine or human milk. Preferably the lactoferrin is isolated from milk by cation exchange chromatography followed by ultrafiltration and diafiltration.

### 3. Isolation of Lactoferrin from Milk

The following is an exemplary procedure for isolating lactoferrin from bovine milk.

Fresh skim milk (7 L, pH 6.5) is passed through a 300 ml column of S Sepharose Fast Flow equilibrated in milli Q water, at a flow rate of 5 ml/min and at 4° C. Unbound protein is washed through with 2.5 bed volumes of water and bound protein eluted stepwise with approximately 2.5 bed volumes each of 0.1 M, 0.35 M, and 1.0 M sodium chloride. Lactoferrin eluting as a discreet pink band in 1 M sodium chloride is collected as a single fraction and dialysed against milli Q water followed by freeze-drying. The freeze-dried powder is dissolved in 25 mM sodium phosphate buffer, pH 6.5 and subjected to rechromatography on S Sepharose Fast Flow with a sodium chloride gradient to 1 M in the above buffer and at a flow rate of 3 ml/min. Fractions containing lactoferrin of sufficient purity as determined by gel electrophoresis and reversed phase HPLC are combined, dialyzed and freeze-dried. Final purification of lactoferrin is accomplished by gel filtration on Sephacryl 300 in 80 mM dipotassium phosphate, pH 8.6, containing 0.15 M potassium chloride. Selected fractions are combined, dialyzed against milli Q water, and freeze-dried. The purity of this preparation is greater than 95% as indicated by HPLC analysis and by the spectral ratio values (280 nm/465 nm) of 19 or less for the iron-saturated form of lactoferrin.

### 4. Metal Ion Saturation or Depletion of Lactoferrin

Iron saturation is achieved by addition of a 2:1 molar excess of 5 mM ferric nitrilotriacetate (Foley and Bates (1987)) to a 1% solution of the purified lactoferrin in 50 mM Tris, pH 7.8 containing 10 mM sodium bicarbonate. Excess ferric nitrilotriacetate is removed by dialysis against 100 volumes of milli Q water (twice renewed) for a total of 20 hours at 4° C. The iron-loaded (holo-) lactoferrin may then be freeze-dried.

Iron-depleted (apo-) lactoferrin is prepared by dialysis of a 1% solution of the highly purified lactoferrin sample in water against 30 volumes of 0.1 M citric acid, pH 2.3, containing 500 mg/L disodium EDTA, for 30 h at 4° C. (Masson and Heremans (1966)). Citrate and EDTA are then removed by dialysis against 30 volumes of milli Q water (once renewed) and the resulting colourless solution may be freeze-dried.

A lactoferrin polypeptide can contain an iron ion (as in a naturally occurring lactoferrin polypeptide) or a non-iron metal ion (e.g., a copper ion, a chromium ion, a cobalt ion, a manganese ion, a zinc ion, or a magnesium ion). For instance, lactoferrin isolated from bovine milk can be depleted of iron and then loaded with another type of metal ion. For example, copper loading can be achieved according to the same method for iron loading described above. For loading lactoferrin with other metal ions, the method of Ainscough, et al. (1979) can be used.

In one embodiment the metal ion is an ion selected from the group comprising aluminium, calcium, copper, chromium, cobalt, gold, iron, manganese, magnesium, platinum, ruthenium, selenium and zinc ions. Preferably the metal ion is an iron ion.

In a preparation of a composition for use according to the invention, a lactoferrin polypeptide or metal ion-binding lactoferrin fragment can be of a single species, or of different species. For instance, the polypeptides or fragments can each contain a different number of metal ions or a different species of metal ions; or the lengths of the polypeptides can vary, e.g., some are full-length polypeptides and some are fragments, and the fragments can each represent a particular portion of a full-length polypeptide. Such a preparation can be obtained from a natural source or by mixing different lactoferrin polypeptide species. For example, a mixture of lactoferrin polypeptides of different lengths can be prepared by proteinase digestion (complete or partial) of full-length lactoferrin polypeptides. The degree of digestion can be controlled according to methods well known in the art, e.g., by manipulating the amount of proteinase or the time of incubation, and described below. A full digestion produces a mixture of various fragments of full-length lactoferrin polypeptides; a partial digestion produces a mixture of full-length lactoferrin polypeptides and various fragments.

### 5. Preparation of Lactoferrin Fragments or Lactoferrin Hydrolysates

Hydrolysates containing candidate functional fragments can be prepared by selecting suitable enzymes with known specificity of cleavage, such as trypsin or chymotrypsin, and controlling/limiting proteolysis by pH, temperature, time of incubation and enzyme to substrate ratio. Refinement of such isolated peptides can be made using specific endopeptidases. As an example, bovine lactoferricin can be produced by cleavage of bovine lactoferrin with pepsin at pH 2.0 for 45 min at 37° C. (Facon & Skura, 1996), or at pH 2.5, 37° C. for 4 h using enzyme at 3% (w/w of substrate) (Tomita et al., 1994). The peptide can then be isolated by reversed phase HPLC (Tomita et al., 1994) or hydrophobic interaction chromatography (Tomita e al., 2002).

Alternatively, lactoferrin peptides can be produced by well established synthetic Fmoc chemistry as described for human kaliocin-1 (NH2-FFSASCVPGADKGQFPNLCRLCAGTGENKCA-COOH) and the lactoferricin derived peptide (NH2-TKCFQWQRNMRKVRGPPVSCIKR-COOH) in Viejo-Diaz et al., (2003); and bovine lactoferricin peptide (NH2-RRWQWRMKKLG-COOH) as described in Nguyen et al., (2005); and lactoferrampin (NH2-WKLLSKAQEKFGKNKSR-COOH) and shorter fragments as described in van der Kraan et al., (2004).

In general, SDS-PAGE may be used to estimate the degree of hydrolysis by comparison of the hydrolysate to a molecular weight standard. Size exclusion chromatography may be used to separate various species within a hydrolysate and to estimate a molecular weight distribution profile.

In a preferred hydrolytic method, bovine lactoferrin was dissolved to 20 mg/mL in 50 mM Tris pH 8.0, 5 mM CaCl₂. Trypsin (Sigma T8642, TPCK treated, Type XII from bovine pancreas, 11700 U/mg protein) was added at an enzyme substrate ratio of 1:50 w/w and the mixture incubated at 25° C. for 3 h. The reaction was stopped by the addition of PMSF to 1 mM final concentration and extent of digestion monitored by SDS-PAGE. The tryptic digest (4 mL) was applied to gel filtration on Sephacryl S300 (Amersham GE) (90 cm×2.6 cm column) in 50 mM Tris, 0.15M NaCl pH 8.0. Suitable fractions containing the major fragments of bovine lactoferrin (Legrand et al., 1984) were then subjected to cation exchange chromatography on S Sepharose fast Flow (Amersham GE) (15 cm×1.6 cm column) using sodium phosphate buffer pH 6.5 and a salt gradient to 1 M NaCl. Final separation of the C lobe and N+C lobes was achieved by further gel filtration on Sephacryl S300 as above but using 10% v/v acetic acid as eluent (Mata et al., 1994). The identity of the dialysed (versus milli-Q water) and freeze-dried fragments was confirmed by SDS-PAGE and Edman N-terminal sequencing.

In another method, a tryptic digest as above was separated by RP-HPLC on a Vydac C18 column as in Superti et al., (2001) and the high mass fragments corresponding to C-lobe and N-lobe fragments recovered. Identity was confirmed by MALDI MS.

In one embodiment hydrolysates useful herein contain one or more functional fragments.

### 6. Immune Enhancement

The present inventors have found that metal ion-saturated lactoferrin is able to stimulate and therefore enhance the immune system. In particular, as shown in the examples below, metal ion-saturated lactoferrin is able to stimulate the generation of antigen-specific cytolytic activity (the activity of immune cells, particularly cytotoxic T-lymphocytes) and/or NK cell activity, improve the cellular immune response to antigens (through the activity of at least cytotoxic T-lymphocytes), improve immune protection (by at least restoring the activity of cytotoxic T-lymphocytes and/or NK cells and enhancing cytokine production), restore immune protection (by at least restoring or stimulating the activity of cytotoxic T-lymphocytes and/or NK cell activity and enhancing cytokine production) and generate pro-inflammatory and immunoregulatory mediators (Th1 and Th2 cytokines). It is believed that any metal ion-saturated functional variant or fragment of lactoferrin will exhibit the same activity as a metal ion-saturated lactoferrin.

Oral iron-saturated bovine lactoferrin induced significant increases in the levels of both Th1 and Th2 cytokines within the tumour and intestine, as shown in the Examples below.

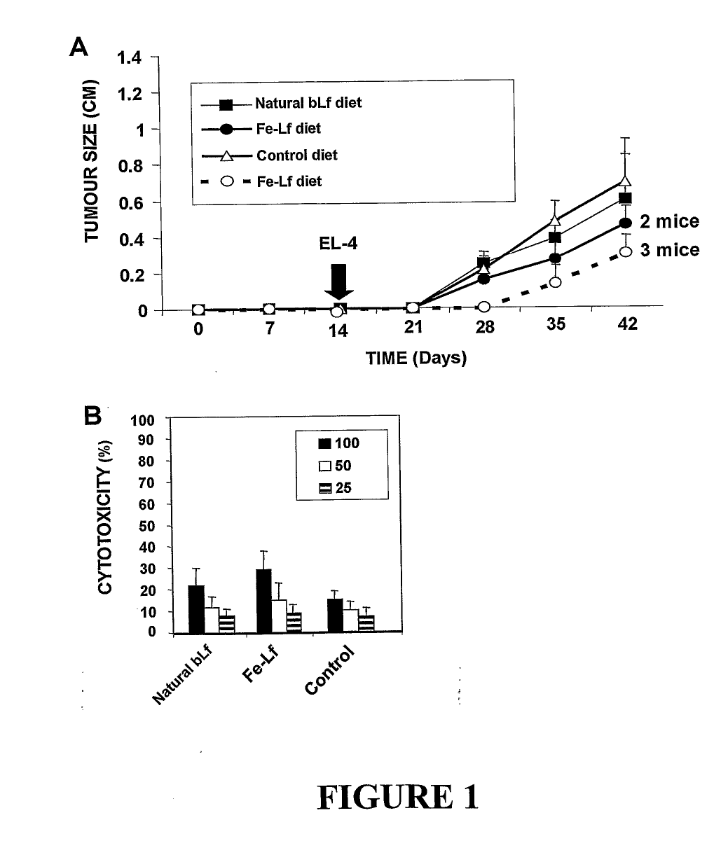

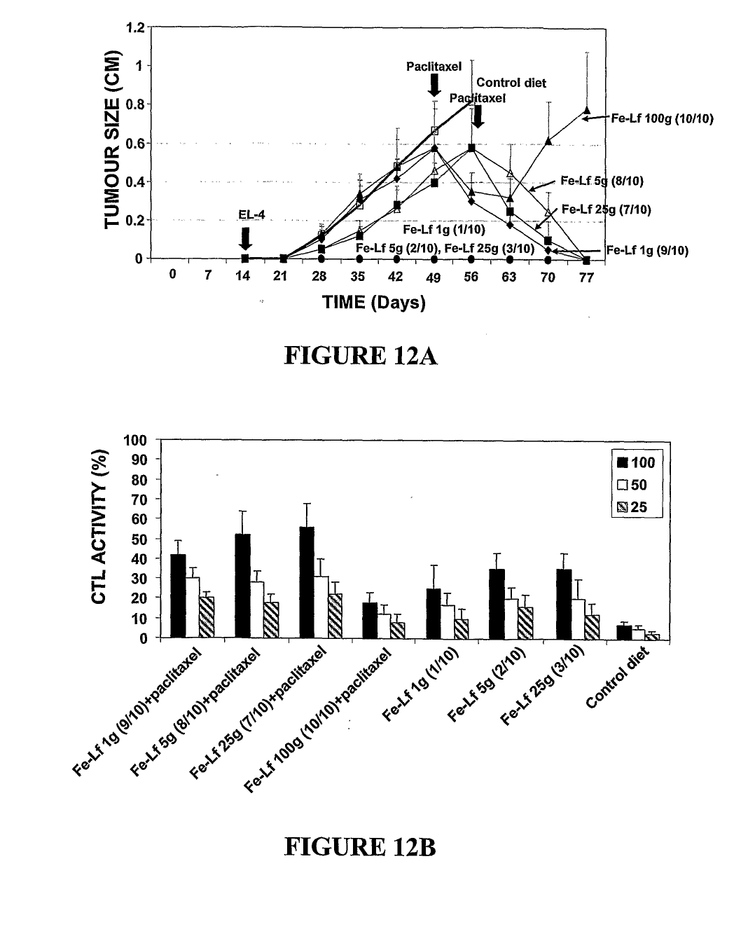

As shown in FIGS. 1A, 1B and 11B, metal ion-saturated lactoferrin is more effective than natural lactoferrin (lactoferrin having an iron saturation of less than 20%, typically 12 to 15%) for improving the generation of antigen-specific cytolytic activity and/or NK cell activity, improving the cellular immune response to antigens, improving immune protection and restoring immune protection. Metal ion-saturated lactoferrin is also more effective than natural lactoferrin at stimulating increased IL-18 production in the gut.

Accordingly, the present invention relates to a method of stimulating the immune system of a subject comprising administration of metal ion-saturated lactoferrin or a metal ion-saturated functional variant or fragment thereof to the subject.

The present invention also relates to a method of increasing the production of Th1 and Th2 cytokines within a tumor of a subject comprising administration of metal ion-saturated lactoferrin or a metal ion-saturated functional variant or fragment thereof to the subject.

The present invention also relates to a method of increasing the production of Th1 and Th2 cytokines within the intestine of a subject comprising administration of metal ion-saturated lactoferrin or a metal ion-saturated functional variant or fragment thereof to the subject.

The present invention also relates to a method of increasing the level of Th1 and Th2 cytokines in the systemic circulation of a subject comprising administration of metal ion-saturated lactoferrin or a metal ion-saturated functional variant or fragment thereof to the subject.

The present invention also relates to a method of increasing an anti-tumour immune response in a subject comprising administration of metal ion-saturated lactoferrin or a metal ion-saturated functional variant or fragment thereof to the subject.

In one embodiment of these methods of the invention, the subject is undergoing or will undergo a cancer therapy as described above.

In one embodiment the subject has a tumour refractory to monotherapy with a chemotherapeutic, anti-angiogenic or immmunotherapeutic agent. In one embodiment the subject has previously undergone unsuccessful monotherapy with a chemotherapeutic, anti-angiogenic or immunotherapeutic agent.

In one embodiment the Th1 cytokine is selected from IL-18, TNF-α and IFN-γ.

In one embodiment the Th2 cytokine is selected from IL-4, IL-5, IL-6 and IL-10. In one embodiment the level of Th1 or Th2 cytokine or cytokines is increased by at least about 100, 150, 200, 250, 300, 350, 400, 450, 500, 550, 600, 650, 700, 750 or 800%

Where appropriate, these methods may be combined with treatments employing any one or more of the anti-tumour agents (including chemotherapeutic agents or immunotherapeutic agents) or anti-tumour therapies described below.

### 7. Haematological Enhancement

The present inventors have found that metal ion-saturated lactoferrin is able to increase white and red blood cell counts. It is believed that any metal ion-saturated functional variant or fragment of lactoferrin will exhibit the same activity as a metal ion-saturated lactoferrin. Accordingly, the present invention relates to a method of maintaining or improving one or both of the white blood cell count and red blood cell count of a subject comprising administration of metal ion-saturated lactoferrin or a metal ion-saturated functional variant or fragment thereof.

This aspect of the invention is useful to increase the red blood cell count of athletes (who are subject to strenuous exercise), increase the red blood cell count of a subject after acute hemorrhage, increase red blood cell count of a subject during or after chemotherapy or radiation treatment, and to increase the red blood cell count of a subject to aid recovery from hemolytic anemia due to drug use, prosthetic heart valve replacement, or serious medical illness (including but not limited to anemia and hepatitis).

In one embodiment the subject is undergoing cancer therapy, preferably chemotherapy, radiation therapy or immunotherapy.

Where appropriate, these methods may be combined with treatments employing any one or more of the anti-tumour agents (including chemotherapeutic agents or immunotherapeutic agents) or anti-tumour therapies described below.

In one embodiment the subject is undergoing treatment with a cytotoxic agent.

### 8. Cancer Prevention

The present inventors have found that metal ion-saturated lactoferrin is able to inhibit tumour formation and inhibit tumour growth. Metal ion-saturated lactoferrin releases anti-tumour factors such as T-cells and/or NK (natural killer) cells and apoptosis-inducing factors into systemic circulation, displays immune enhancing activity, anti-angiogenic activity and direct tumour cytotoxicity, and is able to induce apoptosis of tumour cells as shown in the examples below. It is believed that any metal ion-saturated functional variant or fragment of lactoferrin will exhibit the same activity as a metal ion-saturated lactoferrin.

The present invention has utility in preventing cancer, particularly in preventing relapse (tumour growth) after surgery such as often results from growth and proliferation of secondary tumours, preventing tumour spread after diagnosis and preparing subjects for administration of an anti-tumour agent or anti-tumour therapy.

The utility of the methods of the present invention in preventing cancer lies in the ability of metal ion-saturated lactoferrin to inhibit tumour formation, induce apoptosis, particularly apoptosis of tumour cells, and inhibit angiogenesis, particularly tumour angiogenesis.

Solid tumours must form new blood vessels before they are able to grow beyond a certain size. Therefore, inhibiting angiogenesis, particularly tumour angiogenesis (blood vessel formation to supply tumours) has clear applications in treating cancer (Dass, 2004). As shown in the Examples below, orally administered metal ion-saturated lactoferrin is able to significantly reduce the number of vessels in tumours and significantly reduce blood flow.

Inhibiting angiogenesis also has applications in other disorders including but not limited to cardiovascular diseases (atherosclerosis and restenosis for example), chronic inflammation (rheumatoid arthritis and Crohn's disease for example), diabetes (diabetic retinopathy), psoriasis, endometriosis, macular degeneration and adiposity. Therefore, metal ion-saturated lactoferrin or a functional variant or fragment thereof has applications outside of cancer treatment and prevention.

Similarly, orally administered metal ion-saturated lactoferrin is able to induce apoptosis of tumour cells, as shown in the Examples below. The Examples also show that apoptotic factors are present in blood serum of mice fed metal ion-saturated lactoferrin.

Therefore, the present invention also relates to methods of inhibiting tumour formation in a subject, inducing apoptosis in a subject, inducing apoptosis of tumour cells in a subject, inhibiting angiogenesis in a subject and inhibiting tumour angiogenesis in a subject comprising administration of metal ion-saturated lactoferrin or a metal ion-saturated functional variant or fragment thereof to the subject.

The present invention also relates to a method of maintaining or increasing anti-tumour factors in systemic circulation.

In one embodiment the subject is susceptible to cancer. In one embodiment the subject has a tumour refractory to monotherapy with a chemotherapeutic, anti-angiogenic or immunotherapeutic agent. In one embodiment the subject has previously undergone unsuccessful monotherapy with a chemotherapeutic, anti-angiogenic or immunotherapeutic agent.

Where appropriate, these methods may be combined with treatments employing any one or more of the anti-tumour agents (including chemotherapeutic agents or immunotherapeutic agents) or anti-tumour therapies described below.

### 9. Cancer Treatment and Prevention with Combination Therapies

The present inventors have found that metal ion-saturated, preferably iron-saturated lactoferrin, preferably bovine lactoferrin, is able to inhibit tumour growth and synergizes with immunotherapy (including that mediated by intratumoral gene transfer of B7-1), with chemotherapy (including with paclitaxel, doxorubicin, epirubicin or fluorouracil) or with dendritic cell therapy to substantially eradicate tumours. Metal ion-saturated, preferably iron-saturated lactoferrin, preferably bovine lactoferrin, is able to synergize with chemotherapy (including with paclitaxel, doxorubicin, epirubicin, fluorouracil, cyclophosphamide or methotrexate) to inhibit tumour growth. It is believed that any metal ion-saturated functional variant or fragment of lactoferrin will exhibit the same activity as a metal ion-saturated lactoferrin.

As described above, metal ion-saturated lactoferrin was found to release anti-tumour factors such as T-cells and/or NK (natural killer) cells and apoptosis-inducing factors into systemic circulation, display immune enhancing activity, anti-angiogenic activity and direct tumour cytotoxicity, and the ability to induce apoptosis of tumour cells as shown in the examples below. It is believed that any metal ion-saturated functional variant or fragment of lactoferrin will exhibit the same activity as a metal ion-saturated lactoferrin.

In one embodiment the chemotherapeutic agent is paclitaxel, doxorubicin, epirubicin, fluorouracil, cyclophosphamide or methotrexate.

In addition to the methods described above, the present invention relates to methods of inhibiting tumour growth in a subject and methods of treating or preventing cancer in a subject comprising

(a) administration of a metal ion-saturated lactoferrin or a metal ion-saturated functional variant or fragment thereof, and
(b) separate, simultaneous or sequential administration of at least one anti-tumour agent or anti-tumour therapy.

In one embodiment the subject is suffering from or is susceptible to cancer. In one embodiment the subject has a tumour refractory to monotherapy with a chemotherapeutic, anti-angiogenic or immunotherapeutic agent. In one embodiment the subject has previously undergone unsuccessful monotherapy with a chemotherapeutic, anti-angiogenic or immunotherapeutic agent.

In one embodiment the at least one anti-tumour agent is administered orally or parenterally although the preferred route depends on the anti-tumor agent selected. Preferably the at least one anti-tumour agent is administered by intravenous, intraperitoneal or intratumoural injection. Preferably paclitaxel, doxorubicin, epirubicin, fluorouracil, cyclophosphamide and methotrexate are administered by intravenous or intraperitoneal injection. Preferably the expression plasmid encoding B7-1 is administered by intratumoural injection. Alternatively, tumour cells can be harvested from a patient, transfected ex vivo with B7-1 expression plasmid, then transfected cells injected into a patient. Alternatively, soluble B7-Ig fusion protein can be parenterally delivered. Preferably the dendritic cell therapy is administered by intravenous, intraperitoneal, or intratumoural injection.

In one embodiment the metal ion-saturated lactoferrin or a metal ion-saturated functional variant or fragment thereof is administered orally or parenterally.

In one embodiment the metal ion-saturated lactoferrin or metal ion-saturated functional variant or fragment thereof is administered daily for at least about 1, 2, 3, 4, 5, 6, 7, 8, 9 or 10 weeks before administration of the anti-tumour agent or anti-tumour therapy.

In one embodiment the metal ion-saturated lactoferrin or a metal ion-saturated functional variant or fragment thereof is administered for at least about 1, 2, 3, 4, 5, 6, 7, 8, 9, 10, 11, 12, 13, 14, 15, 16, 17, 18, 19, 20 or 21 days or for at least about 1, 2, 3, 4, 5, 6, 7 or 8 weeks or for at least about 1, 2, 3, 4, 5 or 6 months before administration of the anti-tumour agent or the anti-tumour therapy

In one embodiment the metal ion-saturated lactoferrin or a metal ion-saturated functional variant or fragment thereof is administered for at least about 1, 2, 3, 4, 5, 6, 7, 8, 9, 10, 11, 12, 13, 14, 15, 16, 17, 18, 19, 20 or 21 days or for at least about 1, 2, 3, 4, 5, 6, 7 or 8 weeks or for at least about 1, 2, 3, 4, 5 or 6 months after administration of the anti-tumour agent or the anti-tumour therapy has begun.

Preferably the metal ion-saturated lactoferrin or a metal ion-saturated functional variant or fragment thereof is administered at least once daily including continuously over a day by parenteral drip for example.

In one embodiment of a method of the invention the tumour is a large tumour, as described above.

In one embodiment of a method of the invention one or both of the white blood cell count and red blood cell count of the subject is maintained or improved.

In one embodiment the metal ion-saturated lactoferrin or a metal ion-saturated functional variant or fragment thereof is administered in a dosage form comprising digestible protein, preferably casein or other protective protein.

In one embodiment the metal ion-saturated lactoferrin or a metal ion-saturated functional variant or fragment thereof and the at least one anti-tumour agent or anti-tumour therapy provide a synergistic therapeutic effect that is better than the additive effects of either one alone. For example, preferably there is a greater effect on inhibition of tumour formation or growth, tumour regression, cytolytic effects, immune enhancement, generation of Th1 and Th2 cytokines, or the responsiveness of a subject or a tumour to the treatment method.

These methods may be combined with treatments employing any one or more of the anti-tumour agents (including chemotherapeutic agents or immunotherapeutic agents) or anti-tumour therapies described below.

In one embodiment the anti-tumour therapy is selected from therapies such as, but not limited to, surgery, chemotherapies, radiation therapies, hormonal therapies, biological therapies/immunotherapies, anti-angiogenic therapies, cytotoxic therapies, vaccines, nucleic acid-based vaccines (eg nucleic acids expressing a cancer antigen such as DNA vaccines including p185 vaccines), viral-based therapies (eg adeno-associated virus, lentivirus), gene therapies, small molecule inhibitor therapies, nucleotide-based therapies (eg RNAi, antisense, ribozymes etc), antibody-based therapies, oxygen and ozone treatments, embolization, and/or chemoembolization therapies.

In one embodiment the anti-tumour therapy is selected from chemotherapeutic agents including but not limited to topoisomerase inhibitor, alkylating agent, antimetabolite and anthracyclin (a DNA intercalator).

In one embodiment the anti-tumour therapy is selected from chemotherapeutic agents including but not limited to irinotecan (a DNA intercalator), cyclophosphamide (a DNA intercalator), methotrexate, fluorouracil, epirubicin and doxorubicin (a DNA intercalator).

In one embodiment the at least one anti-tumour agent is a chemotherapeutic agent. Preferably the chemotherapeutic agent is selected from tubulin disruptors, DNA intercalators, and mixtures thereof.

Preferred tubulin disruptors include but are not limited to: taxanes such as but not limited to Paclitaxel and Docetaxel, Vinca alkaloids, Discodermolide, Epothilones A and B, Desoxyepothilone, Cryptophycins, Curacin A, Combretastatin A-4-Phosphate, BMS 247550, BMS 184476, BMS 188791, LEP, RPR 109881A, EPO 906, TXD 258, ZD 6126, Vinflunine, LU 103793, Dolastatin 10, E7010, T138067 and T900607, Colchicine, Phenstatin, Chalcones, Indanocine, T138067, Oncocidin, Vincristine, Vinblastine, Vinorelbine, Vinflunine, Halichondrin B, Isohomohalichondrin B, ER-86526, Pironetin, Spongistatin 1, Spiket P, Cryptophycin 1, Dolastatin, Cematodin, Rhizoxin, Sarcodictyin, Eleutherobin, Laulilamide, VP-16 and D-24851.

Preferred DNA intercalators include but are not limited to: Acridines, Actinomycins, Anthracyclines, Benzothiopyranoindazoles, Pixantrone, Crisnatol, Brostallicin, CI-958, doxorubicin (adriamycin), actinomycin D, daunorubicin (daunomycin), bleomycin, idarubicin, mitoxantrone, cyclophosphamide, melphalan, mitomycin C, bizelesin, etoposide, mitoxantrone, SN-38, cis-platin, actinomycin D, amsacrine, DACA, Pyrazoloacridine, Irinotecan and topotecan.

Most preferably the chemotherapeutic agent is paclitaxel, doxorubicin, epirubicin, fluorouracil, cyclophosphamide and methotrexate.

In one embodiment the anti-tumour agent is an immunotherapeutic agent. Preferably the immunotherapeutic agent is an expression plasmid encoding the T cell co-stimulator B7-1, a T cell co-stimulator, or a functionally related molecule, for example a B7-Ig chimera.

In one embodiment the anti-tumour agent or therapy comprises dendritic cell therapy.

In one embodiment the anti-tumour agent comprises one or more angiogenesis inhibitors such as, but not limited to: antiangiogenic antithrombin III; angiostatin; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); Fibronectin fragment; Gro-beta; Halofuginone; heparinase; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16 kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-(TSP-1); TNP-470; Transforming growth factor-beta (TGF-β); Vasculostatin; Vasostatin (calreticulin fragment); ZD6126; ZD 6474; farnesyl transferase inhibitors (FTI); and bisphosphonates.

Additional examples of anti-tumour agents that can be used in the various embodiments of the invention, include, but are not limited to: acivicin; aclarubicin; acodazole hydrochloride; acronine; adozelesin; aldesleukin; alkylating agent; altretamine; ambomycin; ametantrone acetate; aminoglutethimide; amsacrine; anastrozole; anthramycin; antimetabolite; asparaginase; asperlin; azacitidine; azetepa; azotomycin; batimastat; benzodepa; bicalutanide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamnycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; topisomerase inhibitor; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-tumour agents useful herein include, but are not limited to chemotherapeutic agents such as: 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytotoxic factor; cytostatin; dacliximab; decitabine; dehydrodidemnin B; deslorelin; dexamethasone; dexifosfiamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; fiezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin; fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In one embodiment the radiation therapy includes external beam radiation therapy (including gamma-ray and x-ray therapy) and internal radiation therapy using radioisotopes. Radioisotopes may also be used as anti-tumour agents according to the invention.

### 10. Methods of Increasing Tumour Responsiveness to Therapy

The inventors have shown in the Examples below that orally administered metal ion-saturated lactoferrin is able to increase the responsiveness of a subject and increase the sensitivity of a tumour to anti-tumour agents. It is believed that any metal ion-saturated functional variant or fragment of lactoferrin will exhibit the same activity as a metal ion-saturated lactoferrin.

Therefore, the present invention also relates to a method of increasing the responsiveness of a subject to a therapy, such as an anti-cancer therapy selected from the group comprising surgery, chemotherapies, radiation therapies, hormonal therapies, biological therapies/immunotherapies, anti-angiogenic therapies, cytotoxic therapies, vaccines, nucleic acid-based vaccines (eg nucleic acids expressing a cancer antigen such as DNA vaccines including p185 vaccines), viral-based therapies (eg adeno-associated virus, lentivirus), gene therapies, small molecule inhibitor therapies, nucleotide-based therapies (eg RNAi, antisense, ribozymes etc), antibody-based therapies, oxygen and ozone treatments, embolization, and/or chemoembolization therapy comprising administration of metal ion-saturated lactoferrin, a metal ion-saturated functional variant or fragment thereof or a mixture thereof to a subject in need thereof separately, simultaneously or sequentially with the therapy.

The present invention also relates to a method of increasing the sensitivity of a tumour in a subject to a cancer therapy comprising oral or parenteral administration of metal ion-saturated lactoferrin or a metal ion-saturated functional variant or fragment thereof to a subject in need thereof separately, simultaneously or sequentially with administration of the therapy.

Preferably the metal ion-saturated lactoferrin, metal ion-saturated functional variant or fragment thereof or mixture thereof is as described above.

Preferably the therapy is one described above.

These methods may be combined with treatments employing any one or more of the anti-tumour agents (including chemotherapeutic agents or immunotherapeutic agents) or anti-tumour therapies described above.

In one embodiment the metal ion-saturated lactoferrin or metal ion-saturated functional variant or fragment thereof is administered daily for at least about 1, 2, 3, 4, 5, 6, 7, 8, 9 or 10 weeks before administration of the anti-tumour agent or anti-tumour therapy.

In one embodiment the metal ion-saturated lactoferrin or a metal ion-saturated functional variant or fragment thereof is administered for at least about 1, 2, 3, 4, 5, 6, 7, 8, 9, 10, 11, 12, 13, 14, 15, 16, 17, 18, 19, 20 or 21 days or for at least about 1, 2, 3, 4, 5, 6, 7 or 8 weeks or for at least about 1, 2, 3, 4, 5 or 6 months before administration of the anti-tumour agent or the anti-tumour therapy

In one embodiment the metal ion-saturated lactoferrin or a metal ion-saturated functional variant or fragment thereof is administered for at least about 1, 2, 3, 4, 5, 6, 7, 8, 9, 10, 11, 12, 13, 14, 15, 16, 17, 18, 19, 20 or 21 days or for at least about 1, 2, 3, 4, 5, 6, 7 or 8 weeks or for at least about 1, 2, 3, 4, 5 or 6 months after administration of the anti-tumour agent or the anti-tumour therapy has begun.

### 11. Tumour Types

In one embodiment the tumour is, the tumour cells are or the cancer is a leukemia, colon carcinoma, breast cancer, melanoma, skin or lung cancer.

In one embodiment the tumour is, the tumour cells are or the cancer is a leukemia such as but not limited to, acute leukemia, acute lymphocytic leukemia, acute granulocytic leukemia, acute myelocytic leukemia such as myeloblastic, promyelocytic, myelomonocytic, monocytic, erythroleukemia leukemia and myelodysplastic syndrome, chronic leukemia such as but not limited to, chronic myelocytic leukemia, chronic granulocytic leukemia, chronic lymphocytic leukemia, and hairy cell leukemia.

In one embodiment the tumour is, the tumour cells are or the cancer is a lymphoma such as but not limited to Hodgkin's disease and non-Hodgkin's disease.

In one embodiment the tumour is, the tumour cells are from or the cancer comprises a hematopoietic tumor of myeloid lineage such as but not limited to acute and chronic myelogenous leukemia, smoldering multiple myeloma, nonsecretory myeloma and osteosclerotic myeloma.

In one embodiment the tumour is, the tumour cells are from or the cancer comprises a hematopoietic tumor of lymphoid lineage, including leukemia, acute and chronic lymphocytic leukemia, acute and chronic lymphoblastic leukemia, B-cell lymphoma, T-cell lymphoma, Burkitts lymphoma.

In one embodiment the tumour is, the tumour cells are from or the cancer comprises a hematopoietic tumor of B lymphoid lineage, including B-Cell Chronic Lymphocytic Leukemia (B-CLL)/Small Lymphocytic Lymphoma (SLL), Lymphoplasmacytoid Lymphoma, Follicle Center Lymphoma, Follicular Small Cleaved Cell (FSC), Follicular Mixed Cell (FM), Marginal Zone B-cell Lymphoma, Hairy Cell Leukemia, Plasmacytoma/Myeloma B-Cell Prolymphocytic Leukemia (B-PLL), Mantle Cell Lymphoma, Follicle Center Lymphoma, Follicular Small Cleaved Cell (FSC), Follicle Center Lymphoma (follicular large cell), B-Cell Large B-Cell Lymphoma, Precursor B-Lymphoblastic Leukemia/Lymphoma (PB-LBL/L), Burkitt's Lymphoma, High-Grade B-Cell Lymphoma, Burkitt's-like, Small lymphocytic/pro-lymphocytic lymphoma (SLL), Follicular lymphoma (few large cells), Lymphoplasmacytoid lymphoma, Marginal zone lymphoma.

In one embodiment the tumour is, the tumour cells are from or the cancer comprises a hematopoietic tumor of T lymphoid lineage, including Large Granular Lymphocyte Leukemia, Adult T-Cell Leukemia/Lymphoma (ATL/L) [smoldering], Mycosis Fungoides/Sezary Syndrome, T-cell Chronic Lymphocytic Leukemia/Prolymphocytic Leukemia (T-CLL/PLL), Adult T-Cell Leukemia/Lymphoma (ATL/L) [chronic], Angiocentric Lymphoma, Angioimmunoblastic Lymphoma, Peripheral T-Cell Lymphomas, Intestinal T-Cell Lymphoma, Anaplastic Large Cell Lymphoma, Precursor T-lymphoblastic leukemia/lymphoma (T-LBL/L), Adult T-cell leukemia/Lymphoma (ATLL) [acute and lymphomatous], Large granular lymphocyte leukemia, Adult T-cell leukemia/lymphoma (ATL/L), Mycosis fungoides/Sezary syndrome.

Additional cancers and related disorders that may be treated or prevented by methods and compositions of the present invention include but are not limited to the following: Leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic leukemia, chronic granulocytic leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fumgating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Additional cancers or other abnormal proliferative diseases may be treated or prevented according to the invention include but are not limited to the following: carcinoma, including that of the liver, spleen heart, lung, small intestine, large intestine, rectum, kidney, brain, bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosacoma, rhabdomyoscarama, and osteosarcoma; and other tumors, including melanoma, xenoderma pegmentosum, keratoactanthoma, seminoma, thyroid follicular cancer and teratocarcinoma.

In specific embodiments, malignancy or dysproliferative changes (such as metaplasias and dysplasias), or hyperproliferative disorders, are treated or prevented in the ovary, bladder, breast, colon, liver, lung, skin, pancreas, or uterus. In other specific embodiments, sarcoma, melanoma, or leukemia is treated or prevented.

### 12. Skin Cancer Treatment or Prevention

A further embodiment of the present invention is a method of treating or preventing skin cancer comprising the step of applying metal ion-saturated lactoferrin or a metal ion-saturated functional variant or fragment thereof in or on the skin, and/or in the vicinity of the tumor.

In a preferred embodiment the skin is predisposed to skin cancer due to sun exposure.

In a preferred embodiment the cancer is a basal cell carcinoma, a squamous cell carcinoma, or a melanoma.

Preferably, the ion-saturated lactoferrin composition is administered topically, either alone or in combination with standard anti-cancer regimens. Administration in the vicinity of the tumor includes administration near or adjacent to the margins of the tumor or directly in the margin area of the tumor. It is envisioned that ion-saturated lactoferrin inhibits carcinogenesis, stimulates anti-tumour immunity in the local tissue, inhibits tumour angiogenesis, and/or is directly tumouricidal (able to inhibit tumour growth). Briefly, ion-saturated lactoferrin in a suitable carrier at strengths of 0.1%, 1%, 5%, or 10% is applied twice a day to at-risk skin or cancerous skin lesion. Size progression of the tumour is monitored through CT scans and tumor markers where available.

Doses and treatment regimes can be informed by undertaking preclinical trials in a suitable animal model of skin cancer. A region of the skin of mice is shaved and treated with topical application of a carcinogen (for example, 7,12-dimethylbenz(a)-anthracene (DMBA)) that may be followed by irradiation with UV-B (Bestak, et al., 1996). Metal ion-saturated lactoferrin may be applied two days after carcinogen treatment or once a cancerous lesion has formed, preferably in the presence of a dermal penetration enhancer (such as 70% laureth sulphate and 30% phenylpiperazine) that could increase skin permeability. Metal ion-saturated lactoferrin is applied twice a day, or as otherwise required, to the skin or cancerous lesion and tumour growth monitored over a period of weeks to months.

Where appropriate, these methods may be combined with treatments employing any one or more of the anti-tumour agents (including chemotherapeutic agents or immunotherapeutic agents) or anti-tumour therapies described above.

### 13. Compositions of the Invention

The metal ion-saturated lactoferrin or a metal ion-saturated functional variant or fragment thereof useful herein may be formulated for administration in any chosen dosage form; for example, as food, drink, food additive, drink additive, dietary supplement, nutritional product, medical food, nutraceutical, medicament or pharmaceutical.

In one embodiment the present invention relates to use of metal ion-saturated lactoferrin or a metal ion-saturated functional variant or fragment thereof in the manufacture of a food, drink, food additive, drink additive, dietary supplement, nutritional product, medical food, nutraceutical, medicament or pharmaceutical. Preferably the composition is formulated for oral or topical administration. Preferably the composition is formulated for oral or parenteral administration. Preferably the composition is for inhibiting tumour growth, inducing apoptosis, inducing apoptosis of tumour cells, treating or preventing cancer, increasing the responsiveness of a subject or the sensitivity of a tumour to a therapy, maintaining or improving one or both of the white blood cell count and red blood cell count of a subject, increasing the production of Th1 and Th2 cytokines within the intestine or a tumour of a subject, or other uses, as described above. Preferably the metal ion-saturated lactoferrin or metal ion-saturated functional variant or fragment thereof is as described above.

In one embodiment the metal ion-saturated lactoferrin or a metal ion-saturated functional variant or fragment thereof is formulated for administration separately, simultaneously or sequentially with at least one anti-tumour agent or anti-tumour therapy described above.

In one embodiment the metal ion-saturated lactoferrin or a metal ion-saturated functional variant or fragment thereof is formulated for coadministration with the at least one anti-tumour agent or anti-tumour therapy described above.

In one embodiment the metal ion-saturated lactoferrin or a metal ion-saturated functional variant or fragment thereof is formulated for sequential administration with the at least one anti-tumour agent or anti-tumour therapy described above.

In one embodiment the metal ion-saturated lactoferrin or a metal ion-saturated functional variant or fragment thereof is included as or is delivered as an adjuvant for the anti-tumour agent or anti-tumour therapy in that the metal ion-saturated lactoferrin or a metal ion-saturated functional variant or fragment thereof enhances or potentiates the effects of the anti-tumour agent or anti-tumour therapy.

In general, for oral administration a dietary (a food, food additive or food supplement for example), nutraceutical or pharmaceutical composition useful herein may be formulated by a skilled worker according to known formulation techniques.

For example, foods, food additives or food supplements comprising lactoferrin for use according to the invention include any edible consumer product which is able to carry protein. Examples of suitable edible consumer products include confectionary products, reconstituted fruit products, snack bars, muesli bars, spreads, dips, diary products including yoghurts and cheeses, drinks including dairy and non-dairy based drinks, milk powders, sports supplements including dairy and non-dairy based sports supplements, food and drink additives such as protein sprinkles and dietary supplement products including daily supplement tablets. Suitable nutraceutical compositions useful herein may be provided in similar forms.

In one embodiment a composition of the invention is a milk fraction. In one embodiment the milk fraction comprises at least about 1, 5, 10, 15, 20, 25, 30, 35, 40, 45, 50, 55, 60, 65, 70, 75, 80, 85, 90, 95 or 99% by weight metal ion-saturated lactoferrin or a metal ion-saturated functional variant or fragment thereof, and useful ranges may be selected from any of these values (for example, from about 1 to about 99% by weight, from about 5 to about 99% by weight, from about 10 to about 99% by weight, from about 15 to about 99% by weight, from about 20 to about 99% by weight, from about 25 to about 99% by weight, from about 30 to about 99% by weight, from about 35 to about 99% by weight, from about 40 to about 99% by weight, from about 45 to about 99% by weight, from about 50 to about 99% by weight, from about 55 to about 99% by weight, from about 60 to about 99% by weight, from about 65 to about 99% by weight, from about 70 to about 99% by weight, from about 75 to about 99% by weight, from about 80 to about 99% by weight, from about 85 to about 99% by weight, from about 90 to about 99% by weight, or from about 95 to about 99% by weight).

A suitable pharmaceutical composition may be formulated with appropriate pharmaceutically acceptable carriers (including excipients and diluents) selected with regard to the intended dosage form and standard pharmaceutical formulation practice. See for example, Remington's Pharmaceutical Sciences, 16th edition, Osol, A. Ed., Mack Publishing Co., 1980.

While the preferred route of administration is oral, it should be understood that any mode of administration may be suitable for any composition of the invention. Therefore, inhalation (nasal or buccal inhalation) and vaginal and rectal administration of any composition of the invention is also contemplated. Intramedullar, epidural, intra-articular, and intra-pleural administration of any composition of the invention is also contemplated.

A dosage form useful herein may be administered orally as a powder, liquid, tablet or capsule. Suitable dosage forms may contain additional agents as required, including emulsifying, antioxidant, flavouring or colouring agents. Dosage forms useful herein may be adapted for immediate, delayed, modified, sustained, pulsed or controlled release of the active components.

Injectable dosage forms may be formulated as liquid solutions or suspensions. Solid forms suitable for solution in, or suspension in, liquid prior to injection may also be prepared. The dosage form may also be emulsified. Metal ion-saturated lactoferrin or a metal ion-saturated functional variant or fragment thereof may be mixed with carriers such as, for example, water, saline, dextrose, glycerol, ethanol, or the like and combinations thereof.

Sustained-release preparations may be prepared. Suitable examples of sustained-release preparations include semi-penneable matrices of solid hydrophobic polymers containing lactoferrin or a functional variant or fragment thereof. The matrices may be in the form of shaped articles, e.g., films, or microcapsules. Examples of sustained-release matrices include polyesters, hydrogels (for example, poly(2-hydroxyethyl-methacrylate), or poly(vinylalcohol)), polylactides (see U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

In one embodiment a method of the invention comprises administration of a mixture of metal ion-saturated lactoferrin and at least one metal ion-saturated functional variant or fragment thereof. Therefore in one embodiment a composition comprises a mixture of metal ion-saturated lactoferrin and at least one metal ion-saturated functional variant or fragment thereof. In alternative embodiment a composition comprises a mixture of metal ion-saturated functional fragments.

A preferred lactoferrin composition for use herein comprises lactoferrin, or at least one functional variant or fragment thereof, or a mixture of lactoferrin and at least one functional variant or fragment thereof. Preferably the lactoferrin is bovine lactoferrin or human lactoferrin. Preferably the composition further comprises a digestible protein such as casein or other protective protein. Preferably the composition comprises about 0.1 to 90 wt % lactoferrin and about 10 to 90 wt % casein or other protective protein. More preferably the composition consists essentially of about 0.5 to 10 wt % lactoferrin and about 10 to 99 wt % casein or other protective protein. Most preferably the composition consists essentially of about 1 wt % lactoferrin and about 20 wt % casein or other protective protein.

Lactoferrin or at least one functional variant or fragment thereof may also be administered by parenteral routes including but not limited to subcutaneous, intravenous, intraperitoneal, intramuscular and intratumoural administration. Preferably lactoferrin is administered parenterally by injection. Those skilled in the art will be able to prepare suitable formulations for parenteral administration without undue experimentation.

In one embodiment the daily dosage range (by any route) is 0.001 to 100 g of metal ion-saturated (preferably iron saturated) lactoferrin per day, preferably 0.1 to 30 g, 0.1 to 40 g, 0.1 to 50 g, 0.1 to 60 g, 0.1 to 70 g or 0.1 to 80 g per day for a 70 kg adult, preferably 10 mg to 1.5 g/kg/day, preferably 50 mg to 500 mg/kg/day. A higher dose is preferred for short-term treatment and prevention and a lower dose for long-term treatment and prevention.

The metal ion-saturated lactoferrin or a metal ion-saturated functional variant or fragment thereof may be used alone or in combination with one or more other therapeutic agents, including those described above. When used in combination with another therapeutic agent the administration of the two agents may be separate, simultaneous or sequential. Simultaneous administration includes the administration of a single dosage form that comprises both agents and the administration of the two agents in separate dosage forms at substantially the same time. Sequential administration includes the administration of the two agents according to different schedules, preferably so that there is an overlap in the periods during which the two agents are provided. Suitable agents with which the compositions of the invention can be co-administered include chemotherapeutic and immunotherapeutic agents, and other suitable agents known in the art. Such agents are preferably administered parenterally, preferably by intravenous, subcutaneous, intramuscular, intraperitoneal, intramedullar, epidural, intradermal, transdermal (topical), transmucosal, intra-articular, and intrapleural, as well as oral, inhalation, vaginal and rectal administration.

Additionally, it is contemplated that a composition in accordance with the invention may be formulated with additional active ingredients which may be of benefit to a subject in particular instances. For example, therapeutic agents that target the same or different facets of the disease process may be used.

As will be appreciated, the dose of the composition administered, the period of administration, and the general administration regime may differ between subjects depending on such variables as the severity of symptoms of a subject, the type of disorder to be treated, the mode of administration chosen, and the age, sex and/or general health of a subject.

It should also be appreciated that administration may include a single daily dose or administration of a number of discrete divided doses as may be appropriate.

It should be understood that a person of ordinary skill in the art will be able without undue experimentation, having regard to that skill and this disclosure, to determine an effective dosage regime (including daily dose and timing of administration) for a given condition.

The present invention also relates to a parenteral unit dosage form comprising metal ion-saturated lactoferrin, a metal ion-saturated functional variant or fragment thereof or a mixture thereof and at least one anti-tumour agent. Preferably the at least one anti-tumour agent is selected from paclitaxel, doxorubicin, epirubicin, fluorouracil, cyclophosphamide, methotrexate, an expression plasmid encoding the T cell co-stimulator B7-1 and dendritic cell therapy. Alternatively the agent is selected from any of those described above. Preferably the metal ion-saturated lactoferrin, metal ion-saturated functional variant or fragment thereof, or mixture thereof is as described above.

The present invention also relates to a dietary, nutraceutical or oral pharmaceutical composition comprising, consisting essentially of or consisting of metal ion-saturated lactoferrin, a metal ion-saturated functional variant or fragment thereof or a mixture thereof and casein or other protective protein. Preferably the composition consists essentially of about 0.1 to 90 wt % lactoferrin and about 10 to 90 wt % casein or other protective protein. More preferably the composition consists essentially of about 0.5 to 10 wt % lactoferrin and about 10 to 99 wt % casein or other protective protein. Most preferably the composition consists essentially of about 1 wt % lactoferrin and about 20 wt % casein or other protective protein. Preferably the metal ion-saturated lactoferrin, metal ion-saturated functional variant or fragment thereof, or mixture thereof is as described above.

Various aspects of the invention will now be illustrated in non-limiting ways by reference to the following examples.

### EXAMPLES

### Mice and Reagents

Eight to nine week old male and female C57BL/6 mice (University of Auckland, New Zealand) were used. Each diet group (n=5 unless otherwise indicated) contained an equal number of male and female mice. Mice were kept in an air-conditioned room with controlled humidity, temperature, and 12 h light:dark cycle. The mouse EL-4 T cell thymic lymphoma, Lewis lung carcinoma (LLC), and B16 melanoma cells (H-2b) were purchased from the American Type Culture Collection (Rockville, Md., USA). They were cultured at 37° C. in DMEM medium (Gibco BRL, Grand Island, N.Y., USA), supplemented with 10% foetal calf serum, 50 U/ml penicillin/streptomycin, 2 mM L-glutamine, 1 mM pyruvate. The expression plasmid B7-1-pCDM8, which contains a 1.2 kb cDNA fragment encoding full-length mouse B7-1 was constructed from a cDNA clone provided by Dr P Linsley, Bristol-Myers-Squibb, Seattle, Wash., USA, and has been described previously (Kanwar, et al., 1999). Palitaxel was obtained from Bristol-Meyers Squibb, Wash., USA, whereas doxorubicin was from Pharmacia-Upjohn, Kalamazoo, Mich., USA. Epirubucin was obtained from Calbiochem, Calif., USA; fluorouracil was from Mayne Pharma Ltd, Victoria, Australia (purchased from Healthcare Logistics, Auckland, New Zealand); cyclophosphamide from Healthcare Logistics, Auckland; and methotrexate from Calbiochem, Calif., USA. Anti-bovine-specific lactoferrin antibodies were obtained from Bethyl Laboratories Inc., Montgomery, Tex., USA and from Hycult Biotechnology, Unden, The Netherlands.

### Lactoferrin Preparation

Bovine lactoferrin was prepared from skim milk (Fonterra Co-Operative Group Limited, New Zealand) using the method of Norris et al (Norris, G E et al., 1989). A SP Big Beads ion exchanger was loaded with skim milk and washed with water. The column was eluted with 0-0.5M NaCl solution and the eluate discarded. The column was then eluted with 0.5-1.0M NaCl and the eluate recovered. The recovered eluate was subjected to UF/DF using a 30 kDa membrane to reduce salts and low molecular weight components. Filtration was continued until the retentate was between 90 and 93% bovine lactoferrin. The lactoferrin extract obtained had natural levels of iron-saturation of approximately 5 to 15% and is referred to “natural bLf” in the following Examples.

Iron-saturated bovine lactoferrin extract (100% saturated) was prepared from natural bLf by the method of Law et al (1997).

### Lactoferrin Treatment

The experimental diets were prepared by Crop & Food Research, Palmerston North, New Zealand using as a base the powdered AIN93G formulation. Casein was used as the protein source in the AIN93G diet, and contained no lactoferrin. It was supplemented in the experimental diets with natural bLf or iron-saturated bovine lactoferrin prepared as described above, such that the total protein content of the diet was unchanged. The diet contained 28 g of iron-saturated bovine lactoferrin or 28 g of natural bLf extract per 2400 g of diet. Fresh diet was provided biweekly, and mice had free access to food and water throughout the study.

### Experimental Tumor Model and Therapies

Tumors were established by s.c. injection of 2×10⁵ EL-4 cells into the left flank of mice, and growth determined by measuring two perpendicular diameters. Animals were euthanased when tumors reached more than 1.0 cm in diameter, in accord with Animal Ethics Approval (University of Auckland). All experiments included 5 mice per treatment group, unless otherwise indicated. Paclitaxel (30 mg dissolved in 5 ml of Cremophor® EL and dehydrated alcohol) was diluted in 0.9% NaCl and administered i.p. at 30 mg/Kg. Doxorubucin in water was injected i.p. at 15 mg/Kg. The drugs were injected in a volume of 0.01 to 0.02 ml/g body weight. The expression vector encoding mouse B7-1 was prepared by cesium chloride gradient centrifugation, and diluted to 5 μg/μl in a solution of 5% glucose in 0.01% Triton X-100. It was mixed in a ratio of 1:1 (wt:wt) with DOTAP cationic liposomes (Boehringer Mannheim, Germany). Tumors were injected with 180 μl of DNA (180 μg)/liposome complexes, as described previously (Kanwar, et al., 1999 and Kanwar, et al., 2000).

### Measurement of the Generation of Antitumor Cytotoxic T-Lymphocytes (CTLs)

Splenocytes were harvested 28 days following tumour cell injection, as specified. They were incubated at 37° C. with EL-4 target cells in graded E:T ratios in 96-well round-bottom plates. After a 4-hour incubation, 50 μl of supernatant was collected, and lysis was measured using the Cyto Tox 96 Assay Kit (Promega, Madison, Wis., USA). Background controls for non-specific target and effector cell lysis were included. After background subtraction, percentage of cell lysis was calculated using the formula: 100× (experimental-spontaneous effector-spontaneous target/maximum target-spontaneous target).

### Measurement of Apoptosis

For in situ detection of apoptotic cells, tumors were excised and immediately frozen in dry ice, and stored at −70° C. Frozen serial sections of 6-μm thickness were fixed with paraformaldehyde solution (4% in PBS, pH 7.4), and permeabilized with a solution containing 0.1% Triton X-100 and 0.1% sodium citrate. They were incubated with 20 μl TUNEL reagent (In Situ apoptosis detection kit from Boehringer Mannheim, Germany) for 60 min at 37° C., and examined by fluorescence microscopy. Adjacent sections were counter-stained with haematoxylin to count the total number of cells, or the number of apoptotic cells in ten randomly selected fields (magnification of ×40). The apototic index (AI) was calculated as the number of apoptotic cells×100/total number of nucleated cells. For detection of apoptotic cells in vitro, the numbers of apoptotic and necrotic tumour cells were measured by staining with annexin-V-fluos, TUNEL, and trypan blue, as described previously (Kanwar, et al., 2001).

DC Isolation and Fusion with EL-4 Cells

DCs were generated from bone marrow (BM) cultures according to previously described procedures, with slight modification (Inaba, et al., 1992 and Steinman, et al., 2000). DCs (2×10⁷) were mixed with 2×10⁶ HAT-sensitive EL-4 cells and fused together by adding 200 ml of a 50% solution of PEG 4000 (Sigma) in RPMI-1640 medium in a drop-wise fashion over a period of 90 s. As a control, cells were processed as above, but PEG was omitted. Fused cells and controls were cultured at 37° C. in a 5% CO2 atmosphere for 10 to 14 days. The fused cells were dislodged by gentle pipetting, and a single cell suspension made and cloned in 96 wells plates. Cells were characterized by staining for various cell-surface markers, including MHC-I, MHC-II, CD3, B220, CD 11b, CD 11c, CD40, CD80, CD86 and ICAM-1. To quantitate the fusion of DC with EL-4 tumour cells, cells were stained with 5-chloromethylfluorescein diacetate (CMFDA) or 4-chloromethyl benzoyl amino tetramethyl rhodamine (CMTMR) (Molecular Probes, Inc., Eugene, Oreg.)

### DC-EL-4 Hybrid Cell-Based Therapy

Tumors were established by s.c. injection of 2×10⁵ EL-4 cells into the left flank of mice. Three weeks after injection of 2×10⁵ EL-4 cells when tumours reached ˜0.2 cm diameter in size, groups of mice (n=5) were either fed an iron-saturated lactoferrin diet, or maintained on the control diet. When tumours reached ˜0.4 cm in diameter they were injected i.v. with of 1×10⁶ fused DC-EL-4 hybrid cells into the lateral tail vein. A control group of tumour-bearing mice was injected with PBS only. Animals were euthanased when tumors reached more than 1.0 cm in diameter, in accord with Animal Ethics Approval (University of Auckland).

### ELISA of Cytokine and Nitrite Production in the Small Intestine

Cytokine levels in the supernatants of homogenates of the small intestine were determined using a “sandwich” ELISA kit (Endogen, Woburn, Mass.). They were detected within standard curve ranges of 15-375 pg/ml for IL-4, 0-2450 pg/ml for IL-5, 15-1000 pg/ml for TNF-α, 0-3000 pg/ml for IFN-γ, 37-3000 pg/ml for IL-10, 31.3-2000 pg/ml for IL-12, and 31.3-2000 pg/ml for IL-18, respectively. Quantification of nitrite, indicative of NO production, was carried out by the Griess reaction (Sigma, USA). The results are expressed as the mean nitrite concentration (μM)±SD.

### Statistical Analysis

Results were expressed as mean values±standard deviation (S.D.), and a Student's t test was used for evaluating statistical significance. A value of p<0.05 denotes statistical significance, whereas p<0.001 denotes results that are highly significant.

### Example 1

Natural bLf and fully (100%) iron-saturated bovine lactoferrin were fed orally to mice. EL-4 tumour cells (2×10⁵) were injected into the left flank of C57BL/6 mice following two weeks on control AIN93G diet, or the same diet supplemented with either iron-saturated bovine lactoferrin or natural bLf. Iron-saturated bovine lactoferrin slowed the rate of tumour growth such that at day 42 it had caused 31% inhibition (P>0.05) of tumour growth in 2 mice, compared to control mice fed the control diet (FIG. 1A). In 3 mice, it completely prevented tumours from growing for one week, but then tumours appeared and grew at a rate similar to the other 2 mice. Overall, iron-saturated bovine lactoferrin inhibited tumour growth by 51% at day 42 (P<0.05) in the latter 3 mice. In marked contrast, natural bLf only slightly slowed tumour growth, causing a 11% inhibition in tumour size at day 42, compared to control mice fed the control diet. Referring to FIG. 1A, day 0 refers to the day the mice were placed on their diets; tumour size as measured by two perpendicular diameters (in centimetres) was monitored for 28 days; and each point on the graph represents the mean tumour size with 95% confidence intervals for either 5 mice or the number of mice indicated.

Splenocytes were harvested from the mice described in FIG. 1A at day 42 and tested for their cytolytic activity against EL-4 target cells. The anti-tumour CTL and/or NK cell activity of splenocytes obtained from these mice was significantly (P<0.05) increased in animals treated with iron-saturated bovine lactoferrin, and slightly enhanced in response to natural bLf (P>0.05) (FIG. 1B). Referring to FIG. 1B, the percent cytotoxicity is plotted against various effector-to-target cell ratios (E:T ratios); each point represents the mean percent cytotoxicity obtained from 5 mice; and the bar represents 95% confidence intervals.

### Example 2

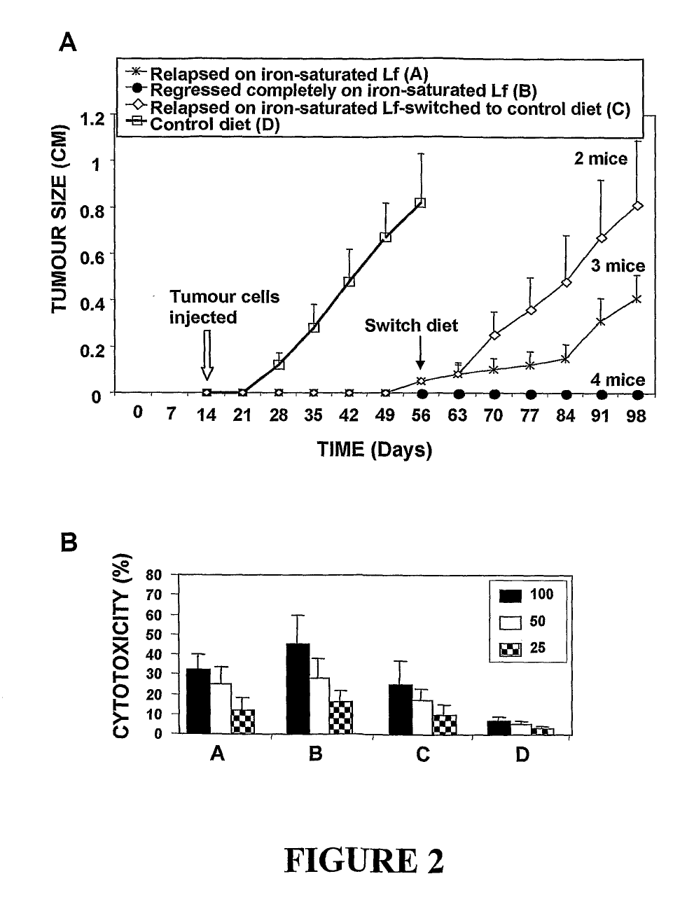

A cohort of 24 mice were fed the control AIN93G diet or the same diet fed supplemented with iron-saturated bovine lactoferrin, and after two weeks on the diets 2×10⁵ EL-4 tumour cells were injected into the left flank of each mouse. Fifteen of the 24 mice developed tumours two weeks later, and were employed in later experiments. Five mice resisted the tumour challenge for 6 weeks, but tumours then appeared and grew at a reduced rate compared to that for tumours of mice fed the control diet (FIG. 2A). One week after tumour appearance, two of these five mice were switched to the control diet (as indicated by the arrow in FIG. 2A). Almost immediately, the growth rate of the tumours of these mice assumed that for tumours of mice fed the control diet. In contrast, the growth rate of tumours in the other three mice maintained on the iron-saturated bovine lactoferrin diet remained suppressed for 4 weeks, but then assumed that for tumours of mice fed the control diet. Four of the original 24 mice remained completely tumour-free for the 11 weeks they were monitored. Referring to FIG. 2A, day 0 refers to the day the mice were placed on their diets; tumour size as measured by two perpendicular diameters (in centimetres) was monitored for 77 days, or alternatively animals were killed when their tumours became larger than 0.8 cm in diameter; each point represents the mean tumour size with 95% confidence intervals for either 5 mice or the number of mice indicated; and the open arrow denotes the day tumour cells were injected.

Splenocytes were harvested from mice in FIG. 2A at day 98 and tested for their cytolytic activity against EL-4 target cells. The anti-tumour CTL and/or NK cell activity of the splenocytes obtained was significantly (P<0.001) increased in all lactoferrin-fed mice, compared to animals maintained on the control diet (FIG. 2B). Nevertheless, there was a direct correlation between anti-tumour CTL and/or NK cell activity generated and the outcome of therapy. Thus, anti-tumour CTL and/or NK cell activity was highest (6-fold increase compared to mice on control diet, P<0.001) for the mice that remained tumour-free, and lowest (30-fold increase compared to mice on control diet, P<0.001) for the mice who developed tumours and were switched from the lactoferrin diet to the control diet. Referring to FIG. 2B, the percent cytotoxicity is plotted against various effector-to-target cell ratios (E:T ratios); each point represents the mean percent cytotoxicity obtained from a group of mice; and the bars represent 95% confidence intervals.

### Example 3

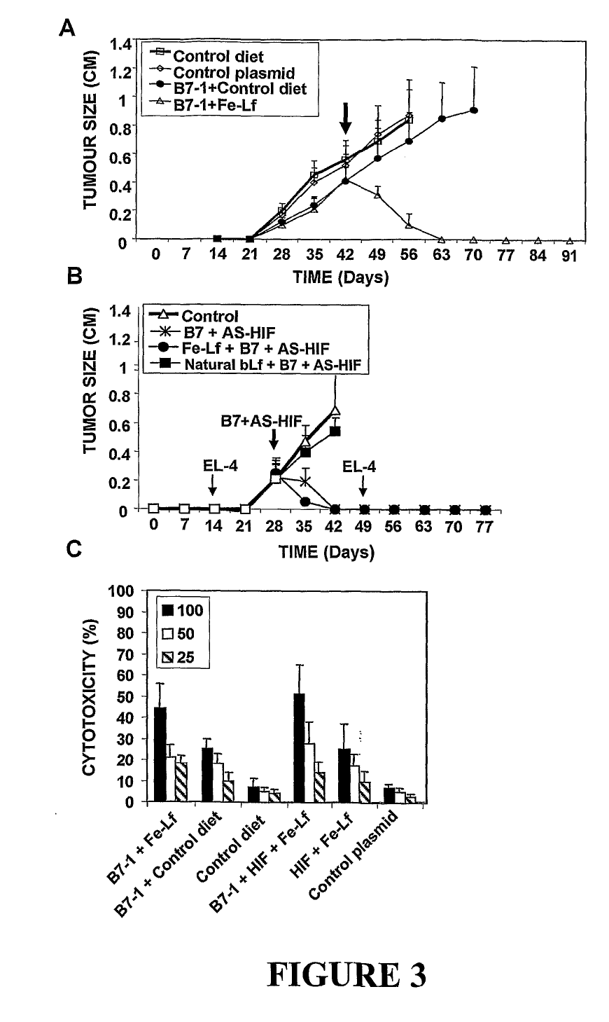

Mice were fed either the base AIN93G diet or the same diet supplemented with iron-saturated bovine lactoferrin for the entire experiment. After 2 weeks on the diets 2×10⁵ EL-4 tumour cells were injected into the left flank of each mouse, and after another 4 weeks tumours that had reached 0.4 cm in diameter were injected with DNA-liposome complexes containing 60 μg of B7-1 expression plasmid. Injection of B7-1 plasmid into the tumours of mice fed the control diet had no detectable affect, and tumours continued to grow unchecked (FIG. 3A). The tumours of mice fed iron-saturated bovine lactoferrin noticeably regressed within one week of delivering the B7-1 plasmid, and completely disappeared after a further two weeks. Thus, iron-saturated bovine lactoferrin potentiates the effects of B7-1 immunotherapy, causing the complete eradication of large immune-resistant tumours. Referring to FIG. 3A, day 0 refers to the day the mice were placed on their diets; the timing of B7-1 administration is indicated by the arrow; tumour size as measured by two perpendicular diameters (in centimetres) was monitored for 91 days, or alternatively animals were killed when their tumours became larger than 0.8 cm in diameter; and each point represents the mean tumour size with 95% confidence intervals for 5 mice.

### Example 4

Mice were fed the control AIN93G diet, or the same diet supplemented with either iron-saturated bovine lactoferrin or natural bLf and tumours were established as described above. Tumour size as measured by two perpendicular diameters (in centimetres) was monitored for 77 days, or alternatively animals were killed when their tumours became larger than 0.75 cm in diameter. Referring to FIG. 3B, day 0 refers to the day the mice were placed on their diets and each point represents the mean tumour size with 95% confidence intervals for 5 mice.

Injection of 60 μg of B7-1 plasmid into EL-4 tumours (0.2 cm diameter) of mice fed the control diet, followed 24 h later by intratumoral injection of 60 μg of antisense HIF-1 plasmid, led to the complete eradication of tumours three weeks later (FIG. 3B).

When the tumours of 2 groups of mice fed the different lactoferrin diets reached ˜0.25 cm in diameter they were injected with DNA-liposome complexes containing 60 μg of a B7-1 expression plasmid, and 2 days later with 60 μg of an expression plasmid encoding antisense HIF-1α. The tumours of mice fed bovine iron-saturated lactoferrin and injected with the combination of B7-1 and antisense HIF-1 plasmids regressed even more rapidly, and also disappeared altogether three weeks later. In marked contrast, the tumors of mice fed a diet supplemented with natural bLf did not respond to B7-1/antisense HIF-1 treatment.

Splenocytes were harvested from mice shown in FIGS. 3A and B at days 56 and 42, respectively, and tested for their cytolytic activity against EL-4 target cells. The percent cytotoxicity is plotted as described previously. Referring to FIG. 3C, B7-1 immunotherapy significantly (P<0.001) enhanced anti-tumour CTL and/or NK cell activity. However, the antitumour CTL and/or NK cell activity of B7-1-treated mice maintained on a diet enriched with iron-saturated bovine lactoferrin was almost doubled compared to mice fed the control diet. Triple treatment of mice with iron-saturated bovine lactoferrin, B7-1, and HIF-1 therapy generated the highest level of anti-tumour CTL and/or NK cell activity (P<0.001).

### Example 5

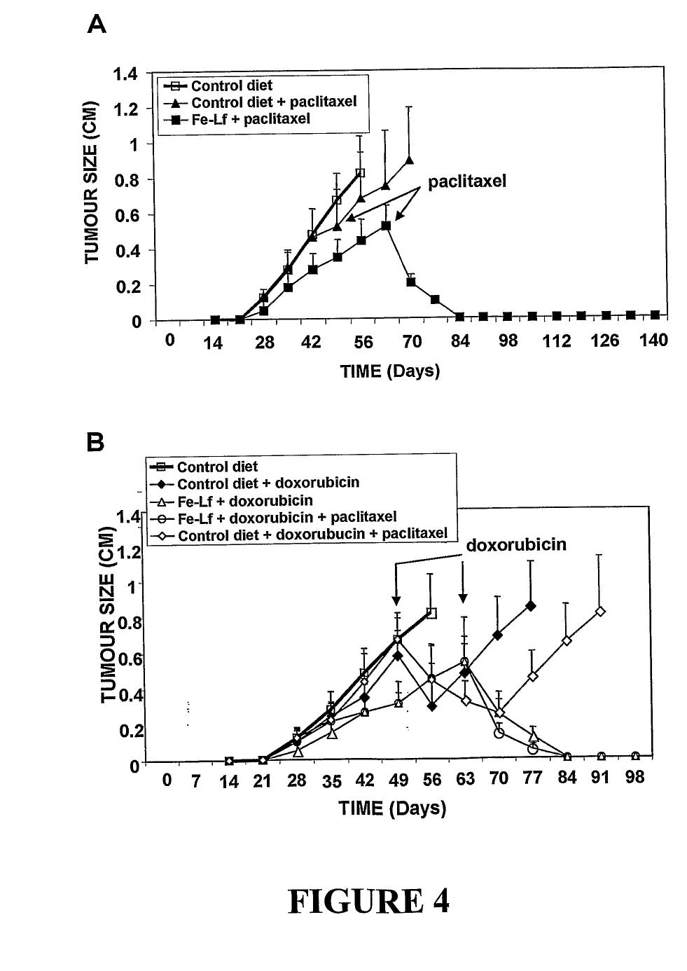

EL-4 tumour cells (2×10⁵) were injected into the left flank of C57BL/6 mice following two weeks on the iron-saturated bovine lactoferrin or control diets. Paclitaxel (30 mg/Kg) was injected i.p. when tumours reached 0.5 cm in diameter. EL-4 tumours of this size grew completely unchecked after paclitaxel treatment in mice fed the control diet (FIG. 4A). In marked contrast, the tumours of mice maintained on an iron-saturated bovine lactoferrin-supplemented diet regressed to less than half their size within one week of administering paclitaxel, and disappeared altogether two weeks later.

### Example 6

EL-4 tumour cells (2×10⁵) were injected into the left flank of C57BL/6 mice following two weeks on the iron-saturated bovine lactoferrin or control diets. Doxorubicin (15 mg/Kg), paclitaxel (30 mg/Kg) or a combination of both was administered as a single dose i.p. when tumours reached ˜0.55 cm in diameter. EL-4 tumours of mice fed the control diet regressed to half their size within one week of administration of doxorubicin, but then began to grow again at a rate identical to tumours of untreated mice fed the control diet (FIG. 4B). In marked contrast, the tumours of mice maintained on an iron-saturated bovine lactoferrin-supplemented diet regressed to less than half their size within one week of administering doxorubucin, and then disappeared altogether two weeks later.

Large EL-4 tumours were resistant to a combination of both doxorubicin and paclitaxel, such that tumours regressed to less than half their size over a three week period following drug administration, but then began to grow again at a rate identical to tumours of untreated mice fed the control diet (FIG. 4B). Once again, oral feeding of iron-saturated bovine lactoferrin potentiated the effect of chemotherapy such that tumours regressed to one-quarter of their size within one week of administering the drug combination, and then disappeared altogether two weeks later (FIG. 4B). The rate of regression was initially more rapid than that achieved with iron-saturated bovine lactoferrin in combination with a single drug.

Referring to FIG. 4B, tumour size was monitored for 98 days, or alternatively animals were killed when their tumours became larger than 0.9 cm in diameter; and each point represents the mean tumour size with 95% confidence intervals for 5 mice.

### Example 7

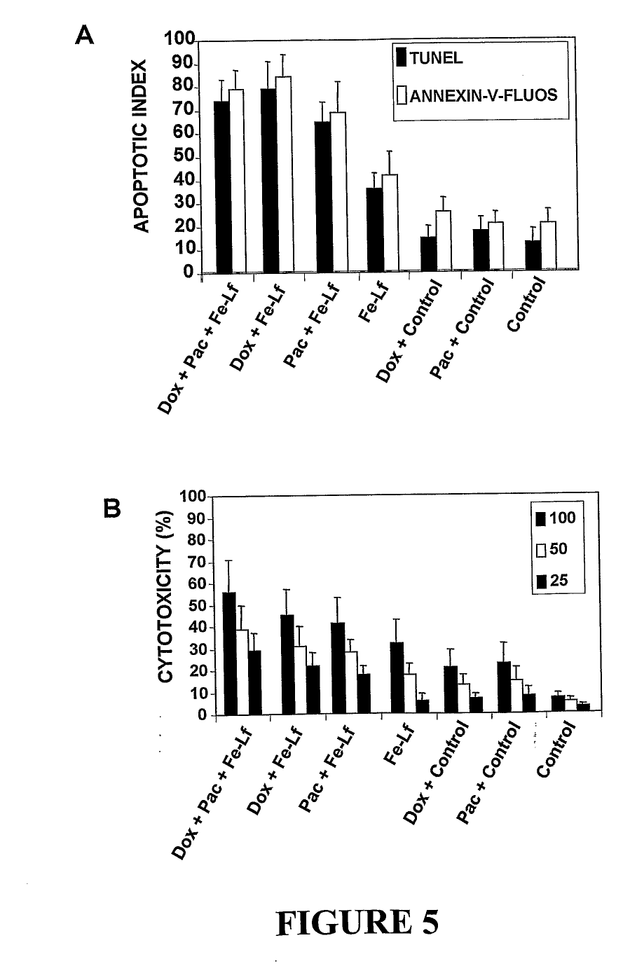

Sections were prepared from tumours of mice shown in FIGS. 4A and 4B at day 56 for the untreated control group fed the control diet, at day 70 for the groups fed either the control or iron-saturated bovine lactoferrin diet receiving paclitaxel and/or doxorubicin, and 7 days after treatment with drugs for mice fed the control diet. Tumour sections were stained by the terminal deoxynucleotidyltransferase-mediated deoxyuridine triphosphate-digoxigenin nick end labeling (TUNEL) method, and also by the annexin-V-fluos method. The number of apoptotic cells detected by TUNEL or annexin-V-fluos staining of tumour sections was determined for 10 randomly selected fields viewed at ×40 magnification. Referring to FIG. 5A, the apoptotic index (A/I) is the number of apoptotic (TUNEL or annexin-V-fluos positive) cells×(100/total number of cells) and bars indicate 95% confidence intervals.

There were surprisingly few apoptotic cells in tumour sections prepared 7 days after administration of the chemotherapeutic drugs in mice fed the control diet (FIG. 5A). Almost identical numbers were observed in the tumour sections (prepared at day 56) of untreated mice maintained on the control diet, in accord with the finding that the chemotherapeutic drugs were largely ineffective. In contrast, there was a 1.9-fold (P<0.001) increase in the apoptotic index for tumours (prepared at day 70) of mice fed iron-saturated bovine lactoferrin compared to mice fed the control diet. The apoptotic index was significantly (P<0.001) increased by 3.7-fold and 5.4-fold, respectively, when lactoferrin-fed mice were treated with paclitaxel or doxorubicin (sections prepared 7 days after administration of the drug). The latter increases in the apoptotic indices correlate with the tumour regression seen in response to the combination treatments.

### Example 8

The anti-tumour CTL and/or NK cell activity of splenocytes obtained from tumour-bearing mice fed a diet supplemented with iron-saturated bovine lactoferrin for 6 weeks was significantly (P<0.001) augmented (5.5-fold increase) versus that of tumour-bearing mice fed the control diet (FIG. 2A), as described previously (FIG. 5B). Monotherapy with each of the chemotherapeutic drugs paclitaxel and doxorubicin stimulated anti-tumor CTL and/or NK cell activity by 3.7-fold (P<0.05) and 3.3-fold (P<0.05) respectively compared to feeding the control diet. Combinational treatments of iron-saturated bovine lactoferrin with paclitaxel, and iron-saturated bovine lactoferrin with doxorubicin, enhanced anti-tumour CTL and/or NK cell activity by 7.3-fold (P<0.001) and 8.2-fold (P<0.001), respectively, at the highest effector:target cell ratio compared to feeding the control diet. The triple combination of iron-saturated bovine lactoferrin, paclitaxel, and doxorubicin increased anti-tumour CTL and/or NK cell generation by 10.3-fold (P<0.05), compared to feeding the control diet. The latter increases in the generation of anti-tumour CTL and/or NK cells correlate with increases in the apoptotic index, and with tumour regression in response to the combination treatments.

### Example 9

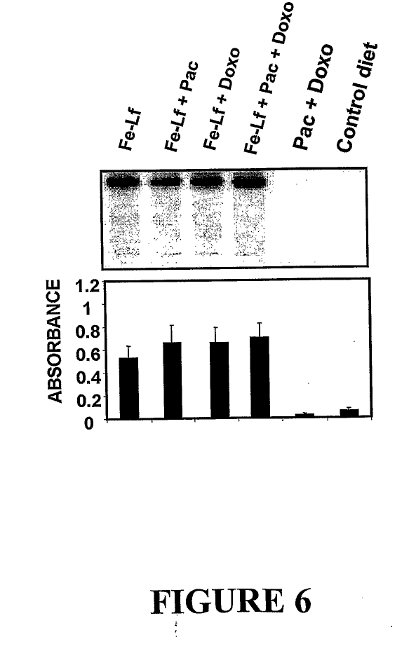

Groups of 5 tumour-bearing mice were fed either the control diet or an iron-saturated bovine lactoferrin diet and treated with paclitaxel or doxorubicin, as indicated. Mice were killed on days described in Example 6 and plasma collected. Referring to FIG. 6, plasma proteins (80 μg) were separated on a 10% polyacrylamide SDS-gel, transferred to a membrane, and blotted with an antibody against bovine lactoferrin (upper panel). The relative amounts of bovine lactoferrin in each lane were recorded by densitometry (lower panel). The order of the plasma samples shown in the histograph is the same for the Western blot. A 75 kDa band characteristic of lactoferrin was present in the plasma samples taken from all the different treatment groups fed bovine lactoferrin, whereas this same band was absent from the plasma of mice fed the control diet (FIG. 6). While there were clear signs of degradation, the bovine lactoferrin in plasma appeared largely undegraded.

### Example 10

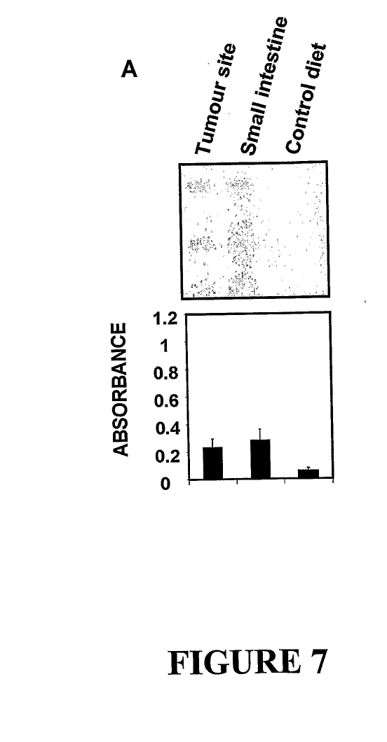

Tumour homogenates and sections were screened to determine whether lactoferrin in the systemic circulation reached the tumour site. A group of 5 tumour-bearing mice were fed the control diet or an iron-saturated bovine lactoferrin diet, and treated with paclitaxel and doxorubicin. Mice were killed on days described in Example 6, and tissues from the tumour and small intestine were collected. Tumour and intestine homogenates (80 μg of protein) were separated on a 10% polyacrylamide SDS-gel, transferred to a membrane, and Western blotted with an antibody against bovine lactoferrin (upper panel of FIG. 7). The relative amounts of bovine lactoferrin in each lane were recorded by densitometry (lower panel of FIG. 7). The order of the plasma samples shown in the histograph is the same for the Western blot.

The 75 kDa band characteristic of lactoferrin, and partially degraded products, were present in homogenates of both the tumour and small intestine, but again were absent from mice fed the control diet (FIG. 7). The anti-bovine antibody heavily-stained small numbers of cells in the tumours of mice fed bovine lactoferrin, which were absent from mice fed the control diet (data not shown).

### Example 11

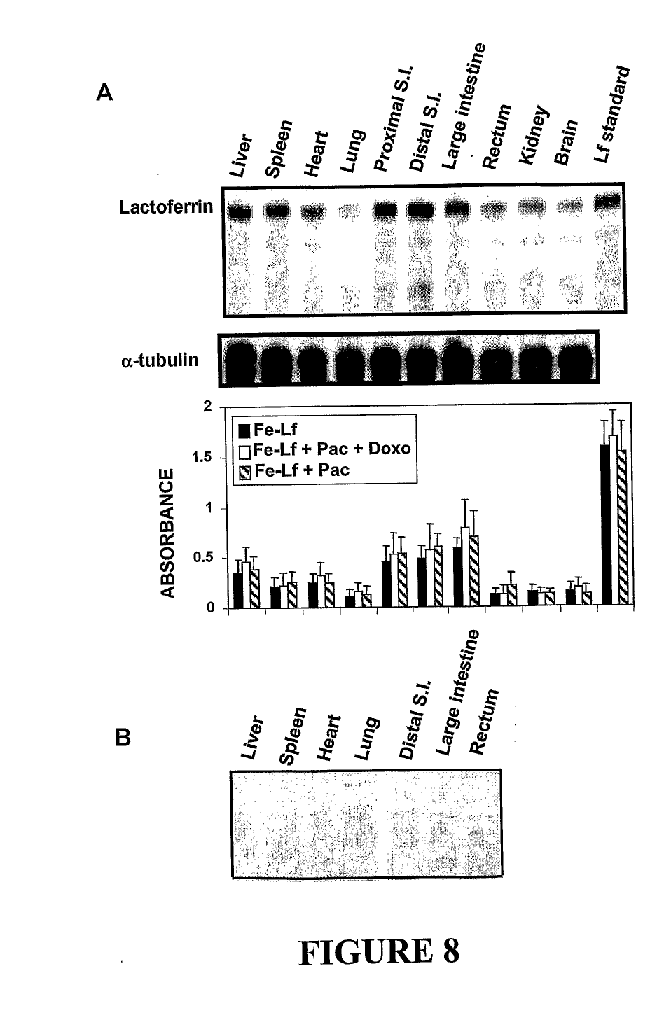

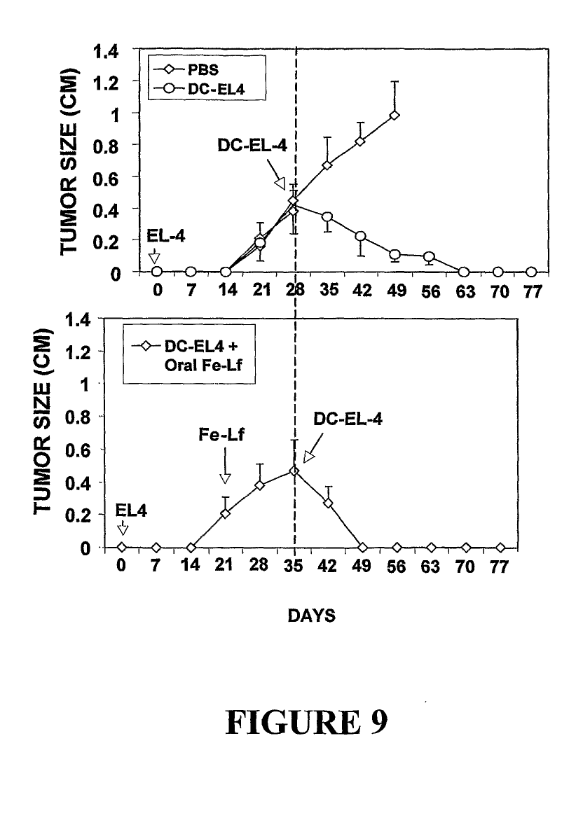

A group of 5 tumour-bearing mice were fed an iron-saturated bovine lactoferrin diet. Mice were killed on days described in Example 6, and tissues from multiple organs, including different regions of the intestine, were collected. Organ homogenates (80 μg of protein) were separated on a 10% polyacrylamide SDS-gel, transferred to a membrane, and blotted with either an antibody against bovine lactoferrin (upper panel of FIG. 8A) or an anti-α-tubulin mAb (middle panel of FIG. 8A). Bovine lactoferrin (1 μg) was included as a standard. The relative amounts of bovine lactoferrin in each lane were recorded by densitometry (lower panel of FIG. 8A). The order of the plasma samples shown in the histograph is the same for the Western blot. Similar results were obtained with mice fed an iron-saturated bovine lactoferrin diet, and treated with paclitaxel, or a combination of paclitaxel and doxorubicin (data not shown). Bovine lactoferrin of 75 kDa was found to be present in all regions of the intestine, and in the liver, but it was also present in lesser amounts in spleen, heart, lung, kidney, and brain (FIG. 8A). Roughly equal amounts of bovine lactoferrin were retained by the proximal and distal regions of the small intestine, and the large intestine.

A group of 5 tumour-bearing mice were fed the AIN93G control diet. Mice were killed on day 56, and tissues from multiple organs, including different regions of the intestine were homogenized and analyzed for bovine lactoferrin expression by Western blot analysis as described above. The 75 kDa band was absent from the intestine and other organs of mice fed the control AIN93G diet (FIG. 8B).

### Example 12

The red and white blood cell counts of mice sacrificed in the preceding examples were recorded. Oral feeding of bovine iron-saturated lactoferrin alone or in combination with administration of paclitaxel or doxorubicin or both increased the mean values of red and white blood cells compared to control mice and compared to mice administered paclitaxel and doxorubicin but not fed lactoferrin, as shown in Table 1.

TABLE 1
Blood haematological profile of control and experimental mice

| | | | | | | Fe-Lf + |
|---|---|---|---|---|---|---|
| | | | Paclitaxel + | Fe-Lf + | Fe-Lf + | paclitaxel + |
| Cells | Control | Fe-Lf | doxorubicin | paclitaxel | doxorubicin | doxorubicin |
| WBCs | 0.5 ± 0.0 | 3.5 ± 1.2 | 0.3 ± 0.1 | 3.7 ± 1.5 | 3.8 ± 1.5 | 3.9 ± 2.0 |
| (×10³/mm³)^{a} | | | | | | |
| RBCs | 4.5 ± 1.5 | 8.2 ± 2.5 | 2.1 ± 1.5 | 8.3 ± 2.5 | 8.5 ± 2.5 | 8.7 ± 3.5 |
| (×10⁶/mm³)^{a} | | | | | | |

^{a}Mean values of red blood cell (RBC) and white blood cell (WBC) counts were recorded in blood samples collected directly from the heart at the time of autopsy.

### Example 13

Tumours were established by s.c. injection of 2×10⁵ EL-4 cells into the left flank of mice. Three weeks after injection of 2×10⁵ EL-4 cells when tumours reached ˜0.2 cm diameter in size, groups of mice (n=5) were either fed an iron-saturated bovine lactoferrin diet, or maintained on the control diet. Tumours in control mice reached ˜0.4 cm in diameter after one week, whereas tumour growth in the lactoferrin-fed mice was delayed such that tumours took two weeks to reach 0.4 cm in diameter. Mice bearing tumours ˜0.4 cm in diameter were injected i.v. with 1×10⁶ fused DC-EL-4 hybrid cells into the lateral tail vein. The tumours of mice fed the control diet and injected with DC-EL-4 hybrids gradually regressed and disappeared altogether five weeks later. In marked contrast, the tumours of mice fed iron-saturated bovine lactoferrin and injected with DC-EL-4 hybrids rapidly regressed taking just two weeks to completely disappear. Tumours grew unchecked in a control group of mice fed the control diet and injected with PBS. Animals were euthanased when tumors reached more than 1.0 cm in diameter.

### Example 14

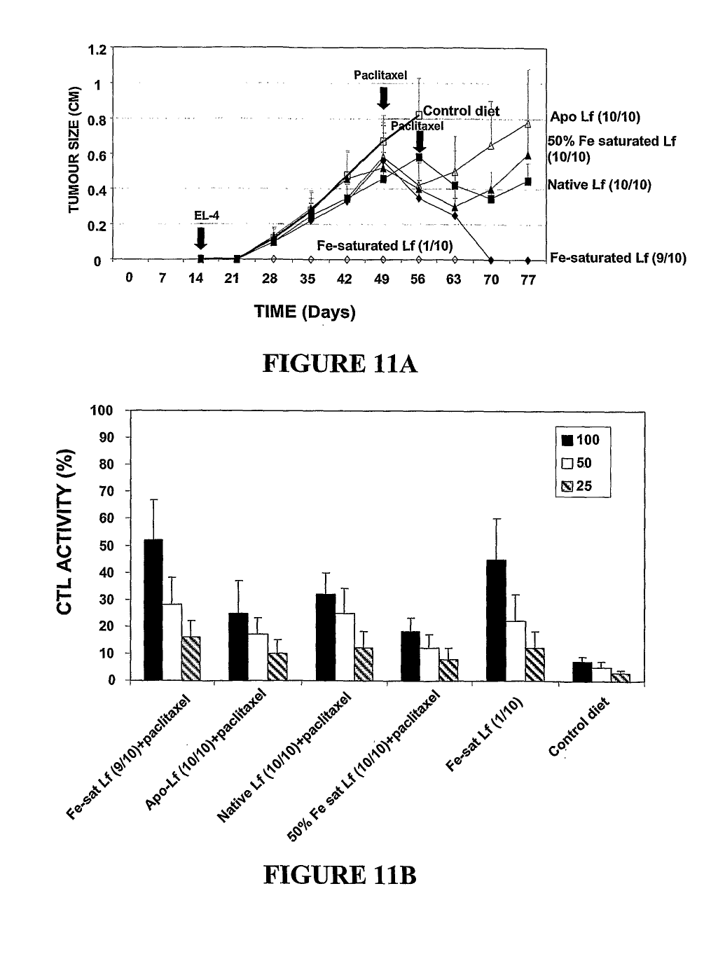

The ability of oral iron-saturated lactoferrin to affect the expression of a panel of Th1 and Th2 cytokines was examined in non-tumour-bearing mice, and in tumour-bearing mice fed iron-saturated bovine lactoferrin or the control diet, and treated with either paclitaxel or doxorubicin or a combination of the two drugs. The levels of both Th1 (IL-18, TNF-α, IFN-γ) and Th2 (IL-4, IL-5, IL-6, IL-10) cytokines increased dramatically (5 to 10-fold, p<0.001) after feeding of iron-saturated bovine lactoferrin, irrespective of whether the mice bore a tumour (FIG. 10). Non-tumour-bearing and tumour-bearing mice displayed similar increases in the expression of IL-4, IL-5, IL-6, IFN-γ and TNF-α, whereas the levels of IL-18 and IL-10 in tumour-bearing mice were less than half those in non-tumour-bearing mice. Both paclitaxel and doxorubicin increased the levels of all cytokines by 5 to 10 fold (P<0.001), and in the case of IL-18 to the same level as that achieved by feeding oral lactoferrin. Paclitaxel and doxorubicin in combination with oral iron-saturated bovine lactoferrin decreased the levels of all cytokines produced in the intestine, with the possible exception of IL-10, and TNF-α to a lesser extent. The levels of each cytokine produced in response to combination therapy were still significantly increased compared those of untreated mice fed the control diet. Iron-saturated bovine lactoferrin increased the presence of all cytokines within the tumor site. Nitrous oxide was increased (3 to 4-fold, P<0.001) both at the tumour site and within the intestine.

### Example 15

Bovine lactoferrin of greater than 90% purity was sourced from the Fonterra Co-operative Group. For the preparation of apo-Lf, a solution of Lf at approximately 80 mg/mL in milliQ water (pH ˜5.7) was adjusted to pH 2.08 by careful addition of 6 M HCl. The solution was stirred at RT for 1 h then dialysed against 10 volumes of 0.1 M citric acid overnight at 4° C. using SpectraPor tubing with a nominal molecular weight cut-off of 3.5 kDa (Spectrum Companies, Ranco Dominguez, Calif., USA). The dialysis fluid was changed twice over a 24 h period, and the Lf solution freeze-dried to a white semi-crystalline powder. For preparation of 50% Fe-saturated lactoferrin, an 8% solution of lactoferrin in 0.1 M sodium bicarbonate was adjusted to pH 8.2 with careful addition of 6 M NaOH. An appropriate volume of 50 mM ferric nitrilo-triacetate (Fe-NTA) (Bates et al., 1967; Brock & Arzabe, 1976) was added to give ˜50% saturation of the lactoferrin (allowing for the purity of the Lf and its native Fe saturation of ˜12%). After stirring for 1 h at RT, the solution (pH 8.01) was dialysed against 10 volumes of milli-Q water overnight at 4° C. using SpectraPor tubing as above. The dialysis fluid was changed twice over a 24 h period and the Lf solution freeze-dried to a salmon red semi-crystalline powder. Lactoferrin of 100% Fe saturation was prepared essentially as for the 50% Fe-saturated material except that the amount of Fe-NTA was adjusted accordingly, and following addition of Fe-NTA, the pH was re-adjusted to 8.0 with careful addition of 6 M NaOH. The final product was a deep salmon red semi-crystalline powder. Fe saturation levels of the final products were verified by spectrophotometric titration (Bates et al., 1967; Brock & Arzabe, 1976). The apo-lactoferrin was approximately 5% Fe-saturated.

A single native lactoferrin preparation was used to generate three additional preparations of lactoferrin, each containing different levels of Fe-saturation. The Fe was removed by citric acid chelation to provide apoLf (5% Fe-saturated), or alternatively lactoferrin was supplemented with Fe to 50% and 100% saturation. Fully Fe-saturated Lf, 50% Fe-saturated Lf, native Lf, and apoLf were fed orally to mice to compare their anti-tumour activities. EL-4 tumour cells (2×10⁵) were injected into the left flank of C57BL/6 mice following two weeks on lactoferrin diets containing 20 g of Lf per 2.4 Kg, or on the control diet. In this particular experiment, the level of Fe-saturation did not appear to effect the growth rate of tumours, except for mice fed the Fe-saturated diet where one of ten mice completely rejected the tumour challenge (FIG. 1A). Paclitaxel (30 mg/Kg) was injected i.p. once tumours reached approximately 0.6 cm in diameter. As before EL-4 tumours of this size were completely resistant to paclitaxel treatment in mice fed the control diet (FIG. 1A). In contrast, the tumours of mice maintained on an iron-saturated bovine Lf-supplemented diet regressed to less than half their size within two weeks of administering paclitaxel, and disappeared altogether a week later (FIG. 1A). The other three preparations of lactoferrin containing lesser levels of Fe were not able to synergize with paclitaxel to eradicate tumours but were still effective to make tumours sensitive to paclitaxel so that tumours were reduced in size. Their efficacy correlated with the degree of Fe-saturation, such that the efficacy of 50% Fe-saturated Lf> native Lf>apoLf. In summary, Fe-saturated Lf, but not lesser Fe-saturated forms of bovine Lf, was able to change a tumour that was completely resistant to chemotherapy into a tumour that was exquisitely sensitive to chemotherapy.

Splenocytes were harvested from the mice described in FIG. 11A at day 77 (or day 56 in the case of controls) and tested for their cytolytic activity against EL-4 target cells. The anti-tumour cytolytic activity of splenocytes obtained from the one mouse fed Fe-saturated lactoferrin which completely resisted the tumour challenge was significantly (P<0.001) increased (by 6-fold), compared to control mice (FIG. 11B). The anti-tumour cytolytic activity of splenocytes was significantly increased in the remaining nine animals treated with fully Fe-saturated Lf (by 6.5-fold, (P<0.001), and to a lesser extent in mice fed 50% Fe-saturated Lf (by 2.5-fold, (P<0.001), native Lf (by 4-fold, (P<0.001), and apoLf (by 3.5-fold, (P<0.001) in combination with paclitaxel treatment. Thus, fully Fe-saturated Lf has the greatest effect in stimulating anti-tumour cytolytic activity in combination with chemotherapy, in accord with the ability of the latter treatment to completely eradicate tumours. Referring to FIG. 11B, the percent cytotoxicity is plotted against various effector-to-target cell ratios (E:T ratios); each point represents the mean percent cytotoxicity obtained from 5 mice; and the bar represents 95% confidence intervals.

### Example 16

Mice were fed the control diet, and the same diet supplemented with different levels of 100% Fe-saturated Lf ranging from 0, 1, 5, 25, and 100 g per 2.4 Kg of diet. EL-4 tumour cells (2×10⁵) were injected into the left flanks of C57BL/6 mice following two weeks on the Lf diets, or control diet. The tumour growth rate of mice fed the lowest and highest doses of Fe-saturated Lf did not differ greatly from that of mice fed the control diet, whereas in contrast, tumours in mice fed diets containing 5 and 25 g of Fe-saturated Lf per 2.4 Kg of diet grew significantly (p<0.05 at days 35-49) more slowly compared to tumours of mice fed the control diet (FIG. 2A). In this particular experiment, one of ten mice fed the 1 g Fe-Lf diet, two of ten mice fed the 5 g Fe-Lf diet, and three of ten mice fed the 25 g Fe-Lf diet completely rejected the tumour challenge. Paclitaxel (30 mg/Kg) was injected i.p. once tumours reached approximately 0.6 cm in diameter. The tumours of mice fed all but the 100 g Fe-Lf diet rapidly regressed and completely disappeared over the following three to four weeks. In contrast, tumours in mice fed the highest dose of Fe-saturated Lf regressed over two weeks, but then re-grew. It was concluded that a diet containing approximately 5 to 25 g of Fe-saturated Lf per 2.4 Kg of diet had the greatest efficacy in inhibiting tumour growth, and rendering tumours susceptible to chemotherapy.

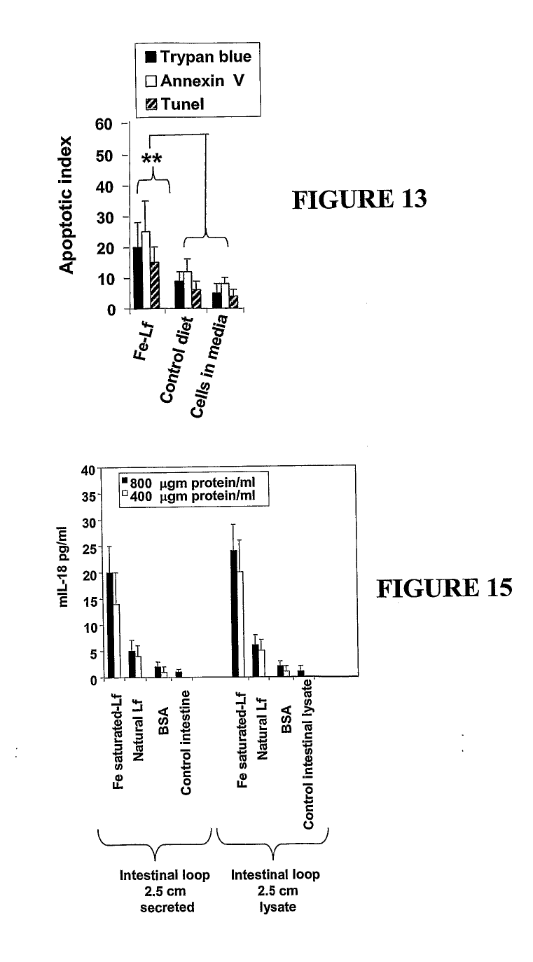

Splenocytes were harvested from the mice described in FIG. 12A at day 77 (or day 56 in the case of controls) and tested for their cytolytic activity against EL-4 target cells. The anti-tumour cytolytic activities of splenocytes obtained from the 6 of 30 mice that rejected the tumour challenge after being fed the 1, 5, and 25 g Fe-saturated Lf diets were significantly increased (by 3 to 4.6-fold, p<0.001) compared to controls (FIG. 12B). The increase in anti-tumour cytolytic activity of splenocytes after injection of tumours with paclitaxel was greatest for mice fed the 5 (6.7-fold, p<0.001) and 25 g (7-fold, p<0.001) Fe-saturated Lf diets, in accord with the ability of the latter treatments to cause rapid and complete tumour regression. In contrast, the increase in anti-tumour cytolytic activity was lowest for mice fed the 100 g Fe-saturated Lf diet (2.2-fold, p<0.001), which did not synergize with paclitaxel to eradicate tumours, although this dose still rendered the tumour susceptible to one dose of paclitaxel. Referring to FIG. 12B, the percent cytotoxicity is plotted against various effector-to-target cell ratios (E:T ratios); each point represents the mean percent cytotoxicity obtained from 5 mice; and the bar represents 95% confidence intervals.

### Example 17

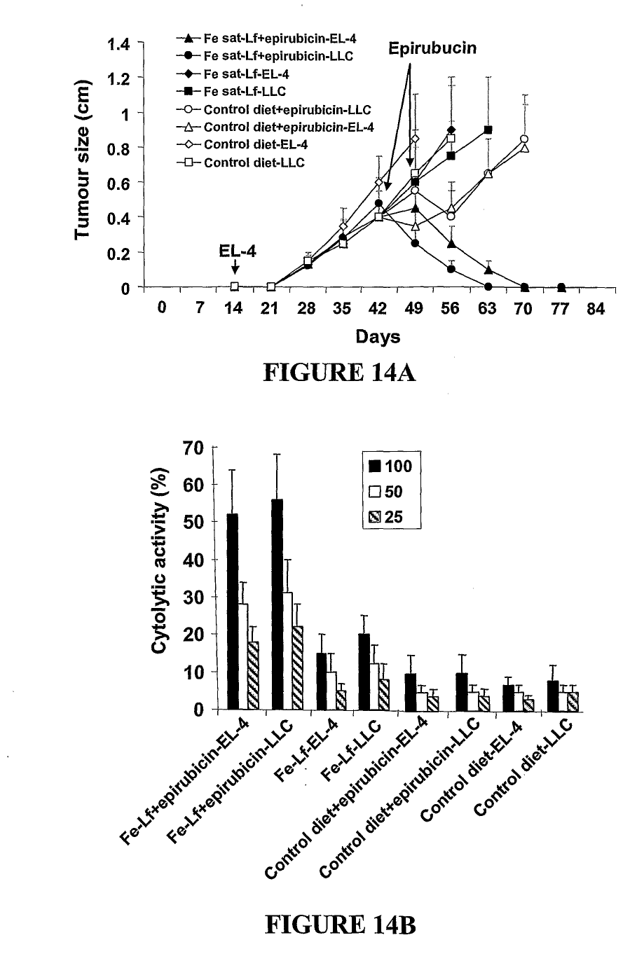

Feeding of Fe-saturated lactoferrin releases anti-tumour factors into the systemic circulation. Fifteen 8 to 9 week-old female C57BL/6 mice were fed the control AIN-93 diet or a diet supplemented with 28 g of 100% Fe-saturated lactoferrin per 2.4 Kg of diet. Sera was collected from experimental and control mice after 6 weeks of feeding the latter diets and tested for their ability to trigger the apoptosis of cultured EL-4 tumour cells. The sera of mice fed the control diet only weakly increased (by 80%) the apoptosis of EL-4 cells in culture, whereas the sera of mice fed Fe-saturated lactoferrin induced a 300% increase (p<0.001) in tumour cell apoptosis, compared to the spontaneous apoptosis of EL-4 cells in culture (FIG. 13).

Sera collected from experimental and control mice was tested for its ability to trigger the apoptosis of cultured EL-4 tumour cells. EL-4 cells (2×10³) in 80 μl of DMEM media were incubated for 24 h in the presence or absence of 100 μl of sera that had been concentrated to 20 μl. The cells were then washed, permeabilized with a solution containing 0.1% Triton X-100 and 0.1% sodium citrate, and incubated with 20 μl of TUNEL reagent (In Situ apoptosis detection kit from Boehringer Mannheim, Germany) for 60 min at 37° C., and examined by fluorescence microscopy. Total numbers of cells were counted by staining the cells with methylene blue. The number of apoptotic cells was counted in ten randomly selected fields (magnification of ×40). The apototic index (AI) was calculated as the number of apoptotic cells×100/total number of nucleated cells. These results were further confirmed by measuring the numbers of apoptotic cells following staining with annexin-V-fluos, and trypan blue.

### Example 18

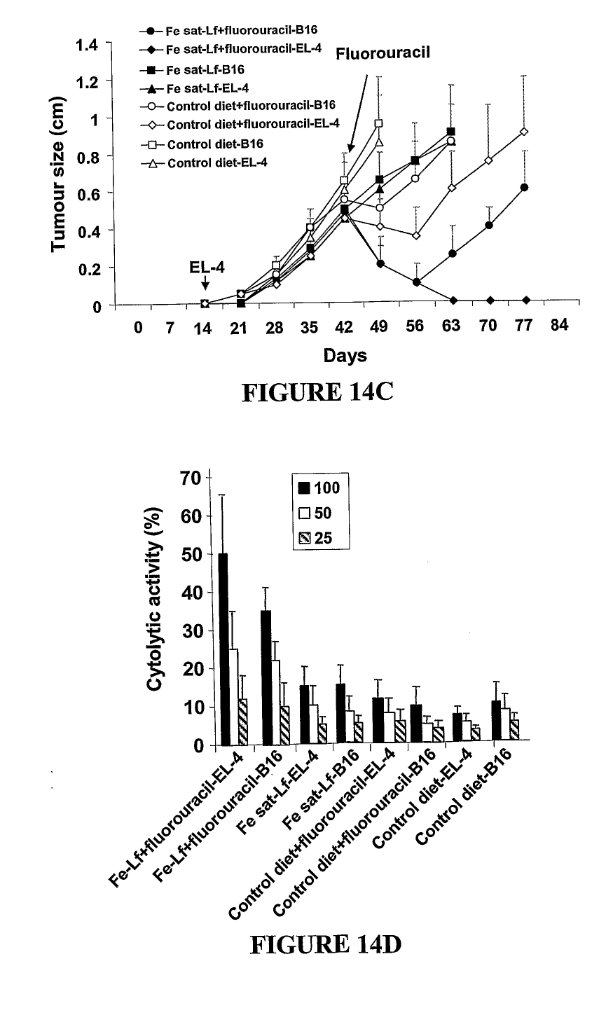

Mice were fed the control diet, and the same diet supplemented with 28 g of 100% Fe-saturated lactoferrin per 2.4 Kg of diet. EL-4 (2×10⁵), Lewis lung carcinoma (LLC, 2×10⁵), and B16 melanoma (2×10⁵) tumour cells were injected into the left flanks of C57BL/6 mice following two weeks on the Lf diets, or control diet. The tumours of mice fed the control diet grew rapidly, reaching 1 cm in diameter within 6 to 7 weeks (FIG. 14A, C, E). Fe-saturated Lf slowed the growth of all three tumour types, but failed to eradicate the tumours altogether. Epirubucin (15 mg/Kg) (FIG. 14A) and fluorouracil (150 mg/Kg) (FIG. 14C) were injected i.p. when tumours reached ˜0.4 to 0.5 cm in diameter. Epirubucin caused a slight delay in the growth of EL-4 and Lewis lung carcinoma tumours of mice fed the control diet, but tumours began to grow rapidly two weeks after drug delivery and thereafter growth continued unabated. In marked contrast, the EL-4 and LLC tumours of mice maintained on an iron-saturated bovine lactoferrin-supplemented diet and treated with epirubucin (FIG. 14A) regressed to half their size within one week of administering the chemotherapeutic agents, and disappeared altogether two weeks later. Similar results were obtained for the EL-4 tumours of mice maintained on an iron-saturated bovine lactoferrin-supplemented diet and treated with fluorouracil (FIG. 14C). With oral Fe-saturated lactoferrin and fluorouracil B16 tumours regressed almost completely over a period of 2 weeks, but then re-grew again (FIG. 14C). Similar results were obtained for EL-4 tumours of mice maintained on an iron-saturated bovine lactoferrin-supplemented diet and treated with cyclophosphamide (100 mg/Kg) (FIG. 14E), but the result was significant given that cyclophosphamide had little effect on the growth of mice fed the control diet. Methotrexate (30 mg/Kg) had no discernible effect on the growth of mice fed the control diet, whereas there was a two week delay in tumour growth when used in combination with a bovine lactoferrin-supplemented diet (FIG. 14E).

Splenocytes were harvested from the mice described in FIGS. 14A and C at day 77 for mice that rejected their tumours, at day 49 or 56 in the case of mice fed the control diet, or when tumours reached 1 cm in diameter in the case of all other tumours. Splenocytes were tested for their cytolytic activity against tumour target cells. The anti-tumour cytolytic activities of splenocytes obtained from the mice fed Fe-saturated Lf and treated with epirubucin that rejected the challenge with EL-4 and LLC tumour cells were significantly increased by 770% (p<0.001) and 590% (p<0.001), respectively compared to controls (FIG. 14B). In contrast, Fe-saturated Lf only increased cytolytic activity by 130% p<0.001) and 150% (p<0.001), respectively compared to controls. Epirubucin had negligible effect in enhancing anti-tumour cytolytic activity. Similarly, the anti-tumour cytolytic activities of splenocytes obtained from the mice fed Fe-saturated Lf and treated with fluoruracil that rejected the challenge with EL-4 and caused the transient regression of B16 tumour cells were significantly increased by 530% (p<0.001) and 220% (p<0.001), respectively compared to controls (FIG. 14D). In contrast, Fe-saturated Lf only increased cytolytic activity by 87% (p<0.05) and 45% (p>0.05), respectively, compared to controls. Fluorouracil only slightly enhanced anti-tumour cytolytic activity. The anti-tumour cytolytic activities of splenocytes obtained from the mice fed Fe-saturated Lf and treated with either cyclophosphamide or methotrexate were increased by only 175% (p<0.001) and 150% (p<0.001), respectively compared to controls (FIG. 14F), in accord with the finding that these combinations were less effective at combating established EL-4 tumours. Nevertheless, anti-tumour cytolytic activity was increased compared to that achieved with Fe-saturated Lf [100% (p<0.05) compared to control]. Cyclophosphamide and methotrexate themselves triggered small increases in anti-tumour cytolytic activity. Referring to FIGS. 14B, D, and F the percent cytotoxicity is plotted against various effector-to-target cell ratios (E:T ratios); each point represents the mean percent cytotoxicity obtained from 5 mice; and the bar represents 95% confidence intervals.

### Example 19

The effects of iron-saturated bovine lactoferrin on tumour blood flow and vascularity were analyzed by perfusion of DiO₇, and by staining of tumour sections with anti-CD31 and anti-CD105 mAbs, respectively. As shown in Table 2, the number of vessels in the tumours of mice fed iron-saturated Lf was significantly reduced by 37-45% (P<0.05), and the blood flow was markedly reduced by 52% (P<0.05), compared to that of mice maintained on the control diet. Paclitaxel and doxorubicin both have anti-angiogenic properties, and in accord analysis of tumours 7 days after administration of each agent revealed reduced tumour vascularity. Paclitaxel significantly reduced the number of vessels and blood flow by 67-72% (P<0.001) and 71% (P<0.001), respectively, whereas doxorubicin reduced the latter by 61-64% (P<0.001) and 65% (P<0.001), respectively, compared to untreated mice fed the control diet. The combinational treatments were only slightly more effective. Thus, the combination of Lf and paclitaxel reduced the number of vessels and blood flow by 73-77% (P<0.001) and 73% (P<0.001), respectively, whereas the combination of Lf and doxorubicin reduced the latter by 66-74% (P<0.001) and 72% (P<0.001), respectively. The triple combination almost completely blocked tumour angiogenesis by reducing the number of vessels and blood flow by 85% (P<0.001) and 85% (P<0.001), respectively.

TABLE 2
Measurement of tumour vascularity and blood flow^{a}

| | Vessel counts per surface area^{b} | | |
|---|---|---|---|
| Treatments | CD31 | CD105 | DiOC7 |
| Control Diet | 32.3 ± 9.9 | 16.5 ± 6.6 | 38.5 ± 8.8 |
| Lf | 20.4 ± 7.7* | 9.0 ± 5.2* | 18.4 ± 8.6* |
| Paclitaxel | 10.5 ± 6.7** | 4.6 ± 3.2** | 11.3 ± 5.3** |
| Lf + paclitaxel | 7.5 ± 3.2** | 4.4 ± 2.2** | 10.4 ± 3.6** |
| Doxorubicin | 11.6 ± 5.3** | 6.5 ± 4.5** | 13.4 ± 6.7** |
| Lf + doxorubicin | 8.5 ± 4.2** | 5.6 ± 3.1** | 10.7 ± 3.9** |
| Lf + paclitaxel + | 5.2 ± 2.2** | 2.4 ± 1** | 5.8 ± 2.5** |
| doxorubicin | | | |

^{a}Tumour blood vessel density was measured x days after feeding the respective diets, or 7 days after injection of paclitaxel and doxorubicin. Tumour sections were stained with the anti-CD31 or anti-CD105 mAbs, or prepared from mice perfused with DiOC₇. A significant difference in mean vessel counts between tumours treated with Lf, paclitaxel, and/or
doxorubicin versus control diet is denoted by an asterisk.
*Indicates a significant difference at P < 0.05, whereas
**indicates a highly significant difference at P < 0.001.
^{b}Values represent means ± SEM, calculated from 5 mice/group.

### Example 20

100% Fe-saturated Lf, natural Lf (less than 20% iron saturated), and bovine serum albumin control were added at 400 and 800 μg/ml to intestinal loops (2.5 cm segments of the small intestine) prepared from healthy 6 to 7 week-old female mice, which were kept in culture for 48 h. IL-18 levels released by the intestinal loops, and present in the supernatants of homogenates of the small intestine were determined using a “sandwich” ELISA kit as described above.

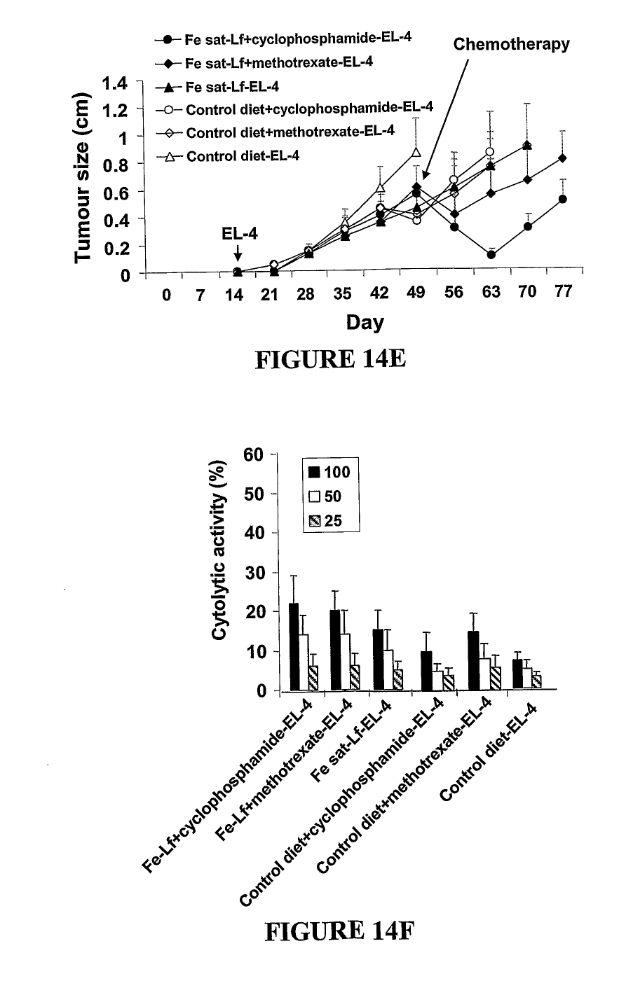

As shown in FIG. 15, Fe-saturated Lf is inherently more active than natural Lf in its ability to stimulate intestinal cytokine production. Thus, incubation of intestinal loops with Fe-saturated Lf led to a 10-fold increase in the levels of IL-18 in the intestine, whereas natural Lf increased the levels of IL-18 by only ˜2-fold, compared to incubation with the bovine serum albumin control protein. The control protein bovine serum albumin had negligible effect on the levels of IL-18 already detectable in the intestine.

### INDUSTRIAL APPLICATION

The methods, medicinal uses and compositions of the present invention have utility in inhibiting tumour growth, maintaining or improving one or both of the white blood cell count and red blood cell count, stimulating the immune system and in treating or preventing cancer. The methods and medicinal uses may be carried out by employing dietary (as foods or food supplements), nutraceutical or pharmaceutical compositions.

Those persons skilled in the art will understand that the above description is provided by way of illustration only and that the invention is not limited thereto.

### REFERENCES

Ainscough, E W, Brodie A, M.; Plowman J E. The chromium, manganese, cobalt and copper complexes of human lactoferrin. Inorganica Chimica Acta 1979, 33 (2) 149-53.
Baker E N, Baker H M, Kidd R., Lactoferrin and transferrin: functional variations on a common structural framework. Biochem Cell Biol. 2002; 80(1):27-34.
Bates G W, Billups C, Saltman P, (1967). The kinetics and mechanism of iron exchange between chelates and transferrin. 1. The complexes of citrate and nitrilotriacetic acid. J. Biol. Chem. 242, 2810-2815.
Bates G W and Schlabach M R (1973). The reaction of Ferric salts with Transferrin. J. Biol. Chem. 248, 3228-3232.
Bestak R, Halliday G M. Sunscreens protect from UV-promoted squamous cell carcinoma in mice chronically irradiated with doses of UV radiation insufficient to cause edema. Photochem Photobiol. 64:188-93, 1996.
Bowie J U, Reidhaar-Olson J F, Lim W A, Sauer R T. Deciphering the message in protein sequences: tolerance to amino acid substitutions. Science. (1990) 247(4948):1306-10.
Brock J H, Arzabe F R (1976), Cleavage of diferric bovine transferrin into two monoferric fragments. FEBS Lett 69, 63-66.
Brock J H. The physiology of lactoferrin. Biochem Cell Biol 2002; 80:1-6.
Cadwell, R. C. and G. F. Joyce. Randomization of genes by PCR mutagenesis. PCR Methods Appl. 1992; 2: 28-33.
Conneely O M. Antiinflammatory activities of lactoferrin. J Am College Nutr 2001; 20:389 S-395S.
Dass C R. Tumour angiogenesis, vascular biology and enhanced drug delivery. J Drug Target. 2004 June; 12(5):245-55
Facon M J, Skura B J. Antibacterial activity of lactoferricin, lysozyme and EDTA against Salmonella enteritidis. Int. Dairy J. 1996, 6 (3) 303-13.
Foley A A, Bates G W. The purification of lactoferrin from human whey to batch extraction Analytica lBiochemistry 1987, 162 (1) 296-300.
Freireich E J, Gehan E A, Rall D P, Schmidt L H, Skipper H E., Quantitative comparison of toxicity of anticancer agents in mouse, rat, hamster, dog, monkey, and man. Cancer Chemother Rep. (1966) 50(4):219-44.
Inaba K, Inaba M, Romani N, Aya H, Deguchi M, Ikehara S, Muramatsu S, Steinman R M. Generation of large numbers of dendritic cells from mouse bone marrow cultures supplemented with granulocyte/macrophage colony-stimulating factor. J Exp Med. 1992; 176:1693-1702.
Kanwar J, Berg R, Lelmert K, and Krissansen G W. Taking lessons from dendritic cells: Multiple xenogeneic ligands for leukocyte integrins have the potential to stimulate anti-tumour immunity. Gene Therapy 1999; 6:1835-44.
Kanwar J R, Kanwar R, Pandey S, Ching L-M, and Krissansen G. W. Vascular attack by 5,6-dimethylxanthenone-4-acetic acid combined with B7.1-mediated immunotherapy overcomes immune-resistance and leads to the eradication of large tumors. Cancer Res 2000; 61:1948-56.
Kanwar J R, Shen W P, Kanwar R K, Berg R W, Krissansen G W. Effects of survivin antagonists on growth of established tumors and B7-1 immunogene therapy. J Natl Cancer Inst. 2001; 93:1541-52.
Kawakami H et al. Effect of lactoferrin on iron solubility under neutral conditions. BiosScience Biotech Biochem 1993; 57(8) 1376-1377.
Kimmerlin T, Seebach D. 100 years of peptide synthesis: ligation methods for peptide and protein synthesis with applications to beta-peptide assemblies. J Pept Res. 2005 February; 65(2):229-60.
S. Karthikeyan, Sujata Sharma, Ashwani K. Sharma, M. Paramasivam, Savita Yadav, A. Srinivasan and Tej P. Singh, Structural variability and functional convergence in lactoferrins, Current Science (1999) 77(2):241
Law, B. A. and Reiter, B., The isolation and bacteriostatic properties of lactoferrin from bovine milk whey. J. Dairy Res. (1977) 44:595-599.
Legrand D, Mazurier J, Metz-Boutigue M-H, Jolles J, Jolles P, Montreuil J & Spik G (1984). Characterization and localization of an iron-binding 18-kDa glycopeptide isolated from the N-terminal half of human lactotransferrin. Biochimica et Biophysica acta 787, 90-96.
Leung, D. W., Chen, E. Y., Goeddel, D. V. A Method for Random Mutagenesis of a Defined DNA Segment Using a Modified Polymerase Chain Reaction. Technique 1989; 1:11-15.
Masson P L, Heremans J F. Studies on lactoferrin, the iron-binding protein of secretions. Protides of the Biological Fluids 1966, 14, 115-24.
Mata L, Castillo H, Sanchez L, Puyol P, Calvo M. Effect of trypsin on bovine lactoferrin and interaction between the fragments under different conditions. J Dairy Res. (1994) 61(3):427-32.
Metz-Boutigue M H, Jolles J, Mazurier J, Schoentgen F, Legrand D, Spik G, Montreuil J,
Jolles P. Human lactotransferrin: amino acid sequence and structural comparisons with other transferrins. Eur J. Biochem. 1984; 145(3):659-76.
Moore S A, Anderson B F, Groom C R, Haridas M, Baker E N. Three-dimensional structure of diferric bovine lactoferrin at 2.8 A resolution. J Mol. Biol. 1997 Nov. 28; 274(2):222-36.
Nguyen L T, Schibli D J, Vogel J. Structural studies and model membrane interactions of two peptides derived from bovine lactoferricin. Journal of Peptide Science 2005, 11 (7) 379-89.
Norris G E, Baker H M, Baker E N. Preliminary crystallographic studies on human apo-lactoferrin in its native and deglycosylated forms. J Mol. Biol. 1989; 209(2):329-31.
Pierce A, Colavizza D, Benaissa M, Maes P, Tartar A, Montreuil J, Spik G. Molecular cloning and sequence analysis of bovine lactotransferrin. Eur J. Biochem. 1991; 196(1):177-84.
Sambrook, J.; Fritsch, E. F.; Maniatis, T. (1989). Molecular Cloning: A Laboratory Manual. Cold Spring Harbour Lab Press, Cold Spring Harbour, N.Y.
Steinman R M, Turley S, Mellman I, Inaba K. The induction of tolerance by dendritic cells that have captured apoptotic cells. J Exp Med. 2000 7; 191:411-416.
Stemmer W P. DNA shuffling by random fragmentation and reassembly: in vitro recombination for molecular evolution. Proc Natl Acad Sci USA 1994; 91:10747-10751.
Sun, X., Kanwar, J. R., Leung, E., Lehnert, K., Wang, D., and Krissansen, G. W. Gene transfer of antisense hypoxia inducible factor-1α enhances the therapeutic efficacy of cancer immunotherapy. Gene Therapy 8: 638-645, 2001.
Superti F, Siciliano R, Rega B, Giansanti F, Valenti P, Antonini G (2001) Involvement of bovine lactoferrin metal saturation, sialic acid and protein fragments in the inhibition of rotavirus infection. Biochim Biophya Acta 1528 (2-3) 107-15.
Tatusova, T. A., Madden, T. L., “Blast 2 sequences—a new tool for comparing protein and nucleotide sequences”, FEMS Microbiol Lett. (1999) 174:247-250.
Tomita M, Takase, M, Wakabayashi H & Bellamy W (1994) Antimicrobial Peptides of Lactoferrin in Lactoferrin Structure and Function, pp 209-218. Eds T W Hutchens, S V Rumball, B Lonnerdal, Plenum Press, New York.
Tomita M, Wakabayashi H, Yamauchi K, Teraguchi S, Hayasawa H; Bovine lactoferrin and lactoferricin derived from milk: production and applications. Biochemistry and Cell Biology 2002, 80 (1) 109-12.
Tomita M, Bellamy W, Takase M, Yamauchi K, Wakabayashi H, Kawasi K. Potent antibacterial peptides generated by pepsin digestion of bovine lactoferrin. J. Dairy Science 1991, 74 (12) 4137-4
Tomita M, Takase M, Bellamy W, Shimamura S. A review: the active peptide of lactoferrin. Acta Paediatr. Jpn. 1994, 36, 585-91.
Tsuda H, Sekine K, Fujita K, ligo M. Cancer prevention by bovine lactoferrin and underlying mechanisms-a review of experimental and clinical studies. Biochem Cell Biol 2002; 131-36.
Tsuji S, Hirata Y, Matsuoka K. Two apparent molecular forms of bovine lactoferrin. J Dairy Sci. 1989; 72(5):1130-6.
van der Kraan M I A, Groenink J, Nazmi K, Veerman E C I, Bolscher J G M, Nieuw Amerongen AV. Lactoferrampin: a novel antimicrobial peptide in the Ni-domain of bovine lactoferrin. Peptides 2004, 25 (2) 177-83.
van Veen H A, Geerts M E, van Berkel P H, Nuijens J H. The role of N-linked glycosylation in the protection of human and bovine lactoferrin against tryptic proteolysis. Eur. J. Biochem. (2004) 271(4): 678-684.
Viejo-Diaz M, Andrés M T, Pérez-Gil J, Sánchez M, Fierro J F. Potassium Efflux Induced by a New Lactoferrin-Derived Peptide Mimicking the Effect of Native Human Lactoferrin on the Bacterial Cytoplasmic Membrane. Biochemistry (Moscow) 2003, 68 (2) 217-27.
Ward P P, Uribe-Luna S, Conneely O M. Lactoferrin and host defense. Biochem Cell Biol 2002; 80:95-102.
Weinberg E D. Human lactoferrin: a novel therapeutic with broad spectrum potential. Pharm Pharmacol 2001; 53:1303-10.
Yoshida, S, and Xiuyn, Ye. Isolation of Lactoperoxidase and Lactoferrins from Bovine Milk Acid Whey by Carboxymethyl Cation Exchange Chromatography. J Dairy Sci. 1991; 74:1439-1444.

1.-8. (canceled)

9. A method of stimulating the immune system of a subject comprising administration of metal ion-saturated lactoferrin or a metal ion-saturated functional variant or fragment thereof to the subject.

10.-13. (canceled)

14. A method of increasing the responsiveness of a subject to a cancer therapy comprising administration of metal ion-saturated lactoferrin or a metal ion-saturated functional variant or fragment thereof to a subject in need thereof separately, simultaneously or sequentially with administration of the therapy.

15.-69. (canceled)

70. The method of claim 14, wherein the administration is oral or parenteral administration.

71. The method of claim 14, further comprising separate, simultaneous or sequential administration of at least one anti-tumour agent or anti-tumour therapy.

72. The method of claim 14, wherein the metal ion-saturated lactoferrin or metal ion-saturated functional variant or fragment thereof is administered daily for at least about 1, 2, 3, 4, 5, 6, 7, 8, 9 or 10 weeks before administration of the anti-tumour agent or anti-tumour therapy.

73. The method of claim 14, wherein the lactoferrin is bovine, human, recombinant bovine or recombinant human lactoferrin.

74. The method of claim 14, wherein the metal ion is an iron ion.

75. The method of claim 14, wherein the metal ion-saturated lactoferrin or metal ion-saturated functional variant or fragment thereof is at least about 25, 30, 35, 40, 45, 50, 55, 60, 65, 70, 75, 80, 85, 90, 91, 92, 93, 94, 95, 96, 97, 98, 99, 99.5 or 100% metal ion-saturated.

76. The method of claim 14, wherein the metal ion-saturated lactoferrin or metal ion-saturated functional variant or fragment thereof is at least about 105, 110, 115, 120, 125, 130, 135, 140, 145, 150, 155, 160, 165, 170, 175, 180, 185, 190, 195 or 200% metal ion-saturated.

77. The method of claim 14, wherein the subject has a leukemia, lymphoma, multiple myeloma, a hematopoietic tumor of lymphoid lineage, a hematopoietic tumor of myeloid lineage, a colon carcinoma, a breast cancer, a melanoma, a skin cancer or a lung cancer.

78. The method of claim 14, wherein the lymphoma, multiple myeloma, a hematopoietic tumor of lymphoid lineage, a hematopoietic tumor of myeloid lineage, a colon carcinoma, a breast cancer, a melanoma, a skin cancer or a lung cancer comprises:
a tumour that is at least about 0.3, 0.4 or 0.5 cm in diameter, or
a tumour that is refractory to monotherapy with one at least one immunotherapeutic, anti-angiogenic or chemotherapeutic agent.

79. A method of claim 14, wherein the cancer therapy is an anti-tumour agent or anti-tumour therapy.

80. A method of claim 79, wherein the anti-tumour therapy is selected from surgery, chemotherapy, radiation therapy, hormonal therapy, biological therapy, immunotherapy, embolization therapy and chemoembolization therapy.

81. A method of claim 79, wherein the anti-tumour agent is a chemotherapeutic agent or an immunotherapeutic agent.

82. The method of claim 9, wherein the stimulation increases the production of Th1 and Th2 cytokines within a tumor of the subject in need thereof.

83. The method of claim 9, wherein the stimulation increases the production of Th1 and Th2 cytokines within the intestine of the subject in need thereof.

84. The method of claim 9, wherein the stimulation increases the level of Th1 and Th2 cytokines in the systemic circulation of the subject in need thereof.

85. The method of claim 9, wherein the stimulation increases an anti-tumour immune response in a subject in need thereof.

86. A method of maintaining or improving one or both of the white blood cell count and red blood cell count of a subject in need thereof comprising administration of metal ion-saturated lactoferrin or a metal ion-saturated functional variant or fragment thereof to the subject.

87. The method of claim 86 wherein the subject has suffered acute haemorrhage, is suffering from haemolytic anemia, has recently undergone strenuous exercise or is undergoing strenuous exercise, therapy for cancer, chemotherapy, radiation therapy, surgery, immunotherapy, or treatment with a cytotoxic agent.


SEQUENCE LISTING (17 sequences)

SEQ ID NO: 1 - protein, 708 aa, Bovine Lactoferrin Precursor
MKLFVPALLSLGALGLCLAAPRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 2 - protein, 689 aa, Bovine Lactoferrin
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 3 - protein, 711 aa, Human Lactoferrin Precursor [sequence omitted]

SEQ ID NO: 4 - protein, 692 aa, Human Lactoferrin
GRRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 5 - protein, 333 aa, Recombinant Human Lactoferrin N-lobe
GRRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLR

SEQ ID NO: 6 - protein, 284 aa, Bovine Lactoferrin N-lobe Fragment
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSR

SEQ ID NO: 7 - protein, 280 aa, Bovine Lactoferrin N-lobe Fragment
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGK

SEQ ID NO: 8 - protein, 281 aa, Bovine Lactoferrin N-lobe Fragment
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKN

SEQ ID NO: 9 - protein, 332 aa, Bovine Lactoferrin N-lobe Fragment
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLR

SEQ ID NO: 10 - protein, 344 aa, Bovine Lactoferrin N-lobe Fragment
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTR

SEQ ID NO: 11 - protein, 681 aa, Bovine Lactoferrin N-terminal Truncation
CTISQPEWFKCRRWQWRMKKLGAPSITCVRRAFALECIRAIAEKKADAVTLDGGMVFEACRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 12 - protein, 405 aa, Bovine Lactoferrin C-lobe Fragment
SFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 13 - protein, 345 aa, Bovine Lactoferrin C-lobe Fragment
VVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 14 - protein, 407 aa, Bovine Lactoferrin C-lobe Fragment
SRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 15 - protein, 348 aa, Bovine Lactoferrin C-lobe Fragment
YTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLKREDFRLLCLDGTRKPVTEAQSCHLAVAPNHAVVSRSDRAAHVEQVLLHQQALFGKNGKNCPDKFCLFKSETKNLLFNDNTECLAKLGGRPTYEEYLGTEYVTAIANLKKCSTSPLLEACAFLTR

SEQ ID NO: 16 - protein, 335 aa, Bovine Lactoferrin C-lobe Fragment
YTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLKREDFRLLCLDGTRKPVTEAQSCHLAVAPNHAVVSRSDRAAHVEQVLLHQQALFGKNGKNCPDKFCLFKSETKNLLFNDNTECLAKLGGRPTYEEYLGTEYVTAIANLKKCS

SEQ ID NO: 17 - protein, 357 aa, Bovine Lactoferrin C-lobe Fragment
ETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR